(19) United States
(12) Patent Application Publication
(10) Pub. No.: US 2022/0259283 A1
(43) Pub. Date: Aug. 18, 2022

(54) SORCS2 CRYSTAL STRUCTURE AND USES THEREOF

(71) Applicant: AARHUS UNIVERSITET, Aarhus C (DK)

(72) Inventors: Joachim POLD VILSTRUP, Aarhus C (DK); Sergio Eduardo Costa ALMEIDA, Risskov (DK); Søren SKOU THIRUP, Aarhus C (DK); Sune SKELDAL, Viby J (DK); Peder SØNDERGAARD MADSEN, Risskov (DK); Simon GLERUP PEDERSEN, Risskov (DK)

(21) Appl. No.: 17/618,155

(22) PCT Filed: Jun. 22, 2020
(86) PCT No.: PCT/EP2020/067315

§ 371 Date: Dec. 10, 2021

(30) Foreign Application Priority Data
Jun. 20, 2019 (EP) 19181521.6

(51) Int. Cl. C07K 14/705; C07K 1/30; G16B 15/30

(52) U.S. Cl. CPC C07K 14/70571; C07K 1/306; A61K 38/00; C07B 2200/13; G16B 15/30

(57) ABSTRACT

The present invention relates to a SorCS2 crystal structure, the atomic coordinates obtained by X-ray crystallography of same, and their use in molecular modelling. The invention further relates to methods of growing crystals of SorCS2. Furthermore, the invention relates to peptides capable of binding to SorCS2, as well as their use as medicament, for example in the treatment of frontotemporal dementia.

### TECHNICAL FIELD

The present invention relates to a SorCS2 crystal structure, the atomic coordinates obtained by X-ray crystallography of same, and their use in molecular modelling. The invention further relates to methods of growing crystals of SorCS2. Furthermore, the invention relates to peptides capable of binding to SorCS2, as well as their use as medicament, for example in the treatment of frontotemporal dementia.

### BACKGROUND

The Vps10p domain receptor family contains five members each of which display distinct trafficking properties and accordingly affect differential biological processes [11]. Whereas sortilin is widely expressed in both neuronal and non-neuronal tissues, expression of SorCS2, another Vps10p domain receptor, mainly prevails in the central nervous system (CNS). SorCS2 play an important role in several cellular processes, which in some cases are involved in various diseases and disorders. However, the underlying molecular mechanisms and structure-function relationships remain to be understood.

Frontotemporal lobar degeneration (FTLD) is the second most common form of dementia but the most common form below the age of 60 and is currently without any treatment. The secreted growth factor progranulin (PGRN) induces cell survival, neurite outgrowth and synapse formation, and plays an important role for the proper functioning and homeostasis of the central nervous system [1]. Reduced plasma levels of progranulin caused by heterozygous loss-of-function mutations in the GRN gene encoding progranulin lead to FTLD [2,3]. A subgroup of these patients additionally exhibits neuronal cytoplasmic inclusions positive for TDP-43 [4].

It is increasingly being recognized that PGRN also plays an important role in lysosomal metabolism [1]. Consistent with this, patients with homozygous loss-of-function mutations in the PGRN gene suffer from a lysosomal storage disorder named neuronal ceroid lipofuscinosis (NCL) [5,6]. Additionally, patients with FTLD and NCL exhibit overlapping cellular pathology characterized by accumulation of lysosomal storage material [7,8].

No signaling receptor for PGRN has been identified, however PGRN is internalized and trafficked to lysosomes by the Vps10p domain receptor sortilin [9]. In lysosomes, progranulin may be proteolytically processed into mature granulins with a possible function in lysosomal metabolism [10].

Crystallographic information on SorCS2 may allow for structure-guided design of compounds capable of binding to SorCS2, and may in turn provide a valuable research tool for development of drugs based on SorCS2 and/or targeting SorCS2.

### SUMMARY

The present inventors have solved the crystal structure of hSorCS2 (SEQ ID NO: 1) in complex with a short peptide encompassing four amino acids of PGRN. This enables the use of the atomic coordinates of the SorCS2 polypeptide for a variety of purposes, such as SorCS2 ligand design, which constitute a significant improvement to the state of the art.

Furthermore, present inventors have surprisingly characterized a physical interaction between hPGRN and hSorCS2. In a cell biological context, the SorCS2:PGRN interaction appears to play a significant and important role. SorCS2 reduces absolute levels of PGRN as well as processed granulins. These findings, together with the solved crystal structure may lay the foundation for development of compounds with capacity to inhibit SorCS2:PGRN interactions, which may increase PGRN and granulin levels specifically in CNS for the benefit of patients suffering from FTLD.

In one aspect, the present disclosure relates to a crystal comprising
a) a polypeptide comprising the amino acid sequence of SEQ ID NO: 1;
b) a biologically active sequence variant of the polypeptide of a), wherein the variant has at least 90% sequence identity to SEQ ID NO: 1; and/or
c) a biologically active fragment comprising at least 500 contiguous amino acids of any of a) through b);
wherein the biological activity is SorCS2 activity.

In one aspect, the present disclosure relates to a method of growing the crystal as defined herein, said method comprising the steps of:
a. providing a protein composition comprising a polypeptide of SEQ ID NO: 1 or a fragment or variant thereof;
b. providing a composition comprising a precipitant;
c. arranging an equilibrium between the protein composition of a with the precipitant composition of b in a container; and
d. obtaining a crystal comprising the polypeptide of SEQ ID NO: 1 or the fragment or variant thereof.

In one aspect, the crystal as defined herein is used for determination of the three dimensional structure of SorCS2 or a fragment or variant thereof.

In one aspect, the present disclosure provides a computer-readable data storage medium comprising a data storage material encoded with at least a portion of the structure coordinates set forth in FIG. 12.

In one aspect, the present disclosure relates to use of atomic coordinates as presented in FIG. 12, or atomic coordinates selected from a three-dimensional structure that deviates from the three-dimensional structure as presented in FIG. 12 by a root mean square deviation over protein backbone atoms of not more than 5 Å, in a method for identifying a ligand capable of binding to a β-propeller domain of SEQ ID NO: 1; and/or a fragment or variant thereof.

In one aspect, the present disclosure provides a method of identifying a ligand capable of binding to a β-propeller domain of SEQ ID NO: 1, and/or a fragment or variant thereof, said method comprising the steps of:
a. Generating the spatial structure β-propeller domain of SEQ ID NO: 1 on a computer screen using atomic coordinates as presented in FIG. 12 or atomic coordinates selected from a three-dimensional structure that deviates from the three-dimensional structure that deviates from the three-dimensional structure presented in FIG. 12 by a root mean square deviation over protein backbone atoms of no more than 5 Å;
b. Generating potential ligands with their spatial structure on the computer screen; and
c. Selecting ligands that can bind to at least 1 amino acid residue of the set of binding interaction sites without steric interference.

In one aspect, the present disclosure provides a computer-based method for identifying a ligand capable of binding to a β-propeller domain of SEQ ID NO: 1, and/or a fragment or variant thereof, said method comprising the steps of:
a. Providing a digital embodiment of a three-dimensional structure from the atomic coordinates as presented in FIG. 12, comprising a propeller domain of SEQ ID NO: 1;
b. Generating a digital embodiment of a potential ligand in a computer; and
c. Selecting a ligand that can bind to at least one amino acid residue of the propeller domain of SEQ ID NO: 1.

In one aspect, the present disclosure provides a computer-assisted method for identifying a ligand capable of binding to a β-propeller domain of SEQ ID NO: 1 (SorCS2), and/or a fragment or variant thereof, using a programmed computer comprising a processor, a data storage system, a data input device, and a data output device, said method comprising the steps of:
a. inputting into the programmed computer through said input device data comprising:
   atomic coordinates of a subset of the atoms of said SorCS2, thereby generating a criteria data set;
   wherein said atomic coordinates are selected form at least part of the atomic coordinates data contained in FIG. 12 or atomic coordinates selected from a three-dimensional structure that deviates from the three-dimensional structure presented from the atomic coordinates as presented in FIG. 12, by a root mean square deviation over protein backbone atoms of not more than 5 Å;
b. comparing, using said processor, the criteria data set to a computer data base of low-molecular weight organic chemical structures and peptide fragments stored in the data storage system; and
c. selecting from said data base, using computer methods, a chemical structure having a portion that is structurally complementary to the criteria data set.

In one aspect, the present disclosure provides a method of identifying a potential ligand of a β-propeller domain of SEQ ID NO: 1, and/or a fragment or variant thereof, said method comprising the steps of:
a. introducing into a computer, information derived from atomic coordinates defining a conformation of the β-propeller domain of SEQ ID NO: 1 (SorCS2), and/or a fragment or variant thereof, based on three-dimensional structure determination, whereby a computer program utilizes or displays on the computer screen the structure of said conformation;
wherein said atomic coordinates are selected from the three-dimensional structure as presented from the atomic coordinates as presented in FIG. 12 or atomic coordinates selected from a three-dimensional structure that deviates from the three-dimensional structure presented from the atomic coordinates as presented in FIG. 12 by a root mean square deviation over protein backbone atoms of not more than 5 Å;
b. generating a three-dimensional representation of the β-propeller domain of SEQ ID NO: 1 by said computer program on a computer screen;
c. superimposing a model of a potential ligand on the representation of said β-propeller domain;
d. assessing the possibility of binding and the absence of steric interference of the potential ligand with the β-propeller domain;
e. incorporating said potential ligand compound in a binding assay of said receptor SorCS2; and
f. determining whether said potential ligand inhibit binding of a competing ligand.

In one embodiment, said competing ligand is progranulin or a fragment thereof. In one embodiment, said progranulin fragment comprises the four most C-terminal amino acid residues of progranulin.

In one aspect, the present disclosure provides a method for identifying a ligand of SorCS2, or a fragment thereof, said method comprising the steps of:
a. Selecting a potential ligand using atomic coordinates in conjunction with computer modelling, wherein said atomic coordinates are the atomic coordinated presented in FIG. 12 or atomic coordinates selected from a three-dimensional structure that deviates from the three-dimensional structure presented from the atomic coordinates as presented in FIG. 12 by a root mean square deviation over protein backbone atoms of not more than 5 Å, by docking potential ligands into a set of binding interaction sites in the β-propeller domain of SEQ ID NO: 1, or a fragment thereof, said binding interaction generated by computer modelling and selecting a potential ligand capable of binding to at least one amino acid residue in said set of binding interaction sites in SorCS2;
b. Providing said potential ligand and said receptor SorCs2;
c. Contacting the potential ligand with said receptor SorCS2; and
d. Detecting binding of said receptor SorCS2 by the potential ligand.

In one aspect, the present disclosure relates to an agent comprising the sequence RQLL (SEQ ID NO: 2), wherein the agent comprises no more than 20 amino acid residues.

In one aspect, the present disclosure relates to said agent for use as a medicament.

In one aspect, the present disclosure relates to said agent for use in a method for treating frontotemporal dementia (FTD), wherein said method comprises administering said compound to a subject in need thereof. In one embodiment, the subject is a mammal, such as a human.

In one aspect, the present disclosure relates to a method for increasing the extracellular levels of progranulin, said method comprising administering an agent as disclosed herein.

### DESCRIPTION OF DRAWINGS

FIG. 1. A: Purification of hSorCS2 from CHO-K1 cells co-purified progranulin as revealed by MALDI MS/MS analysis and MS/MS sequencing. B: The identified peptides are shown in bold underline.

FIG. 2. SorCS2 protein crystals (A) and X-ray diffraction images covering 1° oscillation (B).

FIG. 3. hSorCS2 monomeric structure with the individual domains indicated. Insert: Fo-FC contoured at 3 sigma in green indicating the additional electron density accounting for the binding of the tetra peptide RQLL.

FIG. 4. Sensorgrams obtained from SPR analysis of dilution series of purified human progranulin-6his injected onto a CM5 sensor chip containing immobilized soluble human SorCS2.

FIG. 5. Microscale thermophoresis analysis of binding properties between purified soluble human SorCS2 and progranulin-6his (A), Granulin E (B), and tetrapeptide RQLL (C) with calculated solution dissociation constants affinities indicated.

FIG. 6. SPR sensorgrams showing binding properties of soluble human sortilin (A) and SorCS2 (B) injected onto a CM5 sensor chip containing immobilized progranulin with a C-terminal 6his-tag.

FIG. 7. SRP competition studies showing binding between purified soluble human SorCS2 injected onto a CM5 sensor chip containing immobilized progranulin-6his with or without 1000-fold molar excess of tetrapeptide RQLL.

FIG. 8. Sucrose gradient ultracentrifugation experiments of whole brain preparations of mice show changes in progranulin subcellular distribution by the lack of SorCS2.

FIG. 9. Levels of endogenous progranulin (A) or overexpressed progranulin (B) were decreased after transfection with SorCS2, see Example 8.

FIG. 10. Levels of progranulin C-terminally tagged with EGFP in HEK293 cells in lysate (A) and medium (B) after transfection with SorCS2, sortilin, or empty plasmid (EV), see Example 8. Co-transfection with SorCS2 reduced extracellular and intracellular levels of C-terminally EGFP-tagged progranulin in cells.

FIG. 11. SorCS2 decreases granulin levels in transfected HEK293 cells.

FIG. 12. Structural coordinates of hSorCS2 (SEQ ID NO: 1) in complex with RQLL.

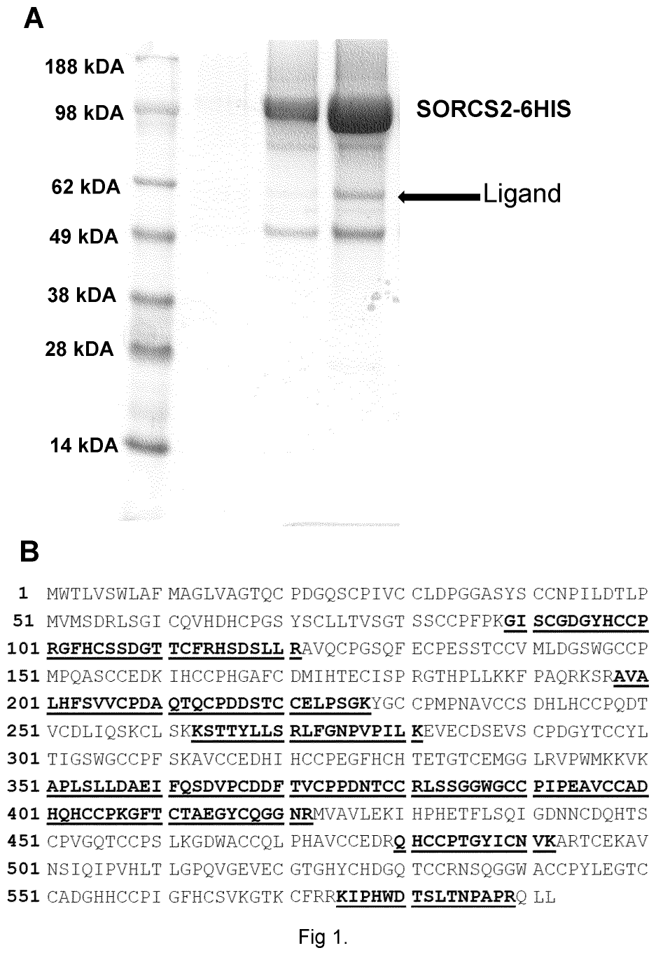

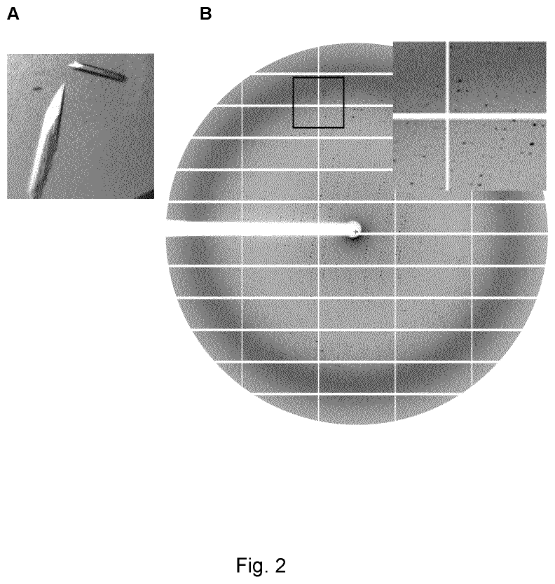

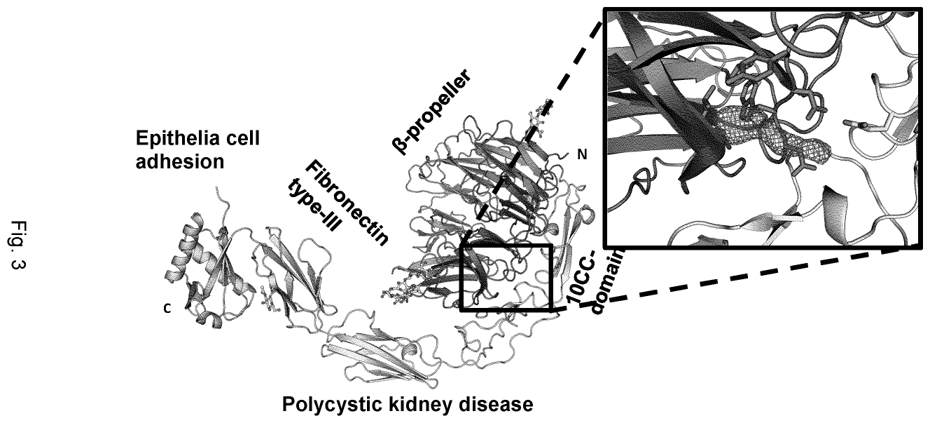

### DETAILED DESCRIPTION

hSorCS2 Crystal

The present inventors have solved the crystal structure of hSorCS2 in complex with a short peptide encompassing four amino acid residues (RQLL) of PGRN, which represents the main binding site of progranulin. Hence, in one aspect, the present disclosure relates to a crystal comprising
d) a polypeptide comprising the amino acid sequence of SEQ ID NO: 1;
e) a biologically active sequence variant of the polypeptide of a), wherein the variant has at least 90% sequence identity to SEQ ID NO: 1; and/or
f) a biologically active fragment comprising at least 500 contiguous amino acids of any of a) through b);
wherein the biological activity is SorCS2 activity.

In one embodiment, the crystal is in complex with at least one ligand. In one embodiment, said at least one ligand is bound to a progranulin binding site of SEQ ID NO: 1. In one embodiment, said progranulin binding site of SEQ ID NO: 1 is in a β-propeller domain of SEQ ID NO: 1. In one embodiment, progranulin binding site of SEQ ID NO: 1 comprises at least one of amino acid residues 528, 459, 468, 475 to 479, 663, and 704 of SEQ ID NO: 1.

In one embodiment, the sequence variant of the polypeptide has at least 91% sequence identity to SEQ ID NO: 1, such as 92% identity, such as 93% identity, such as 94% identity, such as 95% identity, such as 96% identity, such as 97% identity, such as 98% identity, such as 99% identity to SEQ ID NO: 1.

In one embodiment, the amino acid sequence further comprises one or more C-terminal histidine residues.

In one embodiment, the crystal is of orthorhombic of monoclinic space group. For example, the crystal is P12₁1 or P2₁2₁2₁.

In one embodiment, the unit cell parameters are a=137.3±3 Å, b=147.6±3 Å, c=310±3 Å, and α=β=γ=90°. In one embodiment, the unit cell parameters are a=137.3 Å, b=147.6 Å, c=310 Å, and α=β=γ=90°. In one embodiment, the unit cell parameters are a=82.6±3 Å, b=134.6±3 Å, c=146.3247.6±3 Å, and α=90°, β=100.7°, γ=90°. In one embodiment, the unit cell parameters are a=82.6 Å, b=134.6 Å, c=146.3247.6 Å, and α=90°, β=100.7°, γ=90°.

Method of Growing Crystal of hSorCS2

Peptide crystallography involves three main stages: crystallization; data collection and analysis; and generating an electron density map and crystallographic structure. Each of these stages involves a series of steps, starting with the purified peptide and concluding with the deposition of a crystallographic structure into the PDB.

Peptides used for crystallography experiments must be pure and soluble. Impurities, such as peptide fragments generated during peptide synthesis, totaling more than a few percent, may inhibit or prevent crystal growth. The peptide must be water soluble for the techniques described herein, because the peptide is screened in various aqueous solutions containing buffers, salts, and cryogenic protectants.

In one aspect, the present disclosure relates to a method of growing the crystal as defined herein, said method comprising the steps of:
a. providing a protein composition comprising a polypeptide of SEQ ID NO: 1 or a fragment or variant thereof;
b. providing a composition comprising a precipitant;
c. arranging an equilibrium between the protein composition of a with the precipitant composition of b in a container; and
d. obtaining a crystal comprising the polypeptide of SEQ ID NO: 1 or the fragment or variant thereof.

In one embodiment, the temperature within the container is between 1 and 30° C., such as between 10 and 25° C., such as between 15 and 20° C., such as between 18 and 20° C., such as 19° C.

In one embodiment, the protein composition comprises Tris pH 7.2-7.8, such as Tris pH 7.4. In one embodiment, the protein composition comprises in the range of 10 to 100 mM Tris, such as in the range of 10 to 50 mM, such as in the range of 15 to 40 mM, such as in the range of 20 to 30 mM, for example 25 mM Tris. In one embodiment, the protein composition comprises in the range of 10 to 500 mM NaCl, such as in the range of 50 to 250 mM, such as in the range of 100 to 200 mM, such as in the range of 125 to 175 mM, such as 150 mM NaCl.

In one embodiment, the precipitant composition comprises at least one component selected from the group consisting of: sodium citrate, magnesium chloride, and polyethylene glycol (PEG). In one embodiment, the precipitant composition comprises sodium citrate, such as in the range of 10 to 500 mM sodium citrate, for example 100 mM sodium citrate. In one embodiment, the precipitant composition comprises sodium citrate pH 5.5. In one embodiment, the precipitant composition comprises magnesium chloride, such as in the range of 10 to 500 mM magnesium chloride, for example 300 mM magnesium chloride. In one embodiment, the precipitant composition comprises PEG with an average molecular weight from 3350 to 20000 Da. In one embodiment, the precipitant composition comprises PEG selected from the group consisting of: PEG 3350, PEG 4000, PEG 6000, and PEG 8000. In one embodiment, the precipitant composition comprises in the range of 5 to 20% w/v PEG 6000, such as in the range of 7 to 18% w/v PEG 6000, such as in the range of 9 to 16% w/v PEG 6000, such as in the range of 10 to 15% w/v PEG 6000.

In one embodiment, the ratio of volumes between protein composition and precipitant composition is less than 5 to 1, such as less than 4 to 1, such as less than 3 to 1, such as less than 2 to 1, such as 1:1.

In one embodiment, the initial concentration of the polypeptide in the protein composition is between 0.1 to 10 mg/mL, such as between 0.1 and 9 mg/mL, such as between 0.1 and 8 mg/mL, such as between 0.1 and 7 mg/mL, such as between 0.1 and 6 mg/mL, such as between 0.5 and 5 mg/mL, such as between 0.5 and 2.5 mg/mL, such as between 1 to 2 mg/mL.

In one embodiment, said method further comprises the steps of:
a. isolating a crystalline precipitate, and
b. growing the crystalline precipitate by vapor diffusion from hanging drops or sitting drops.

Use of the hSorCS2 Crystal

Solving the crystal structure of hSorCS2 enables the use of the atomic coordinates of the SorCS2 polypeptide for a variety of purposes, such as SorCS2 ligand design. Hence, in one aspect, the crystal as defined herein is used for determination of the three dimensional structure of SorCS2 or a fragment or variant thereof. In one embodiment, the crystal used for determination of the three dimensional structure of SorCS2 further comprises a ligand bound to SorCS2 for determination of the three dimensional structure of SorCS2 or a fragment or variant thereof in complex with said ligand.

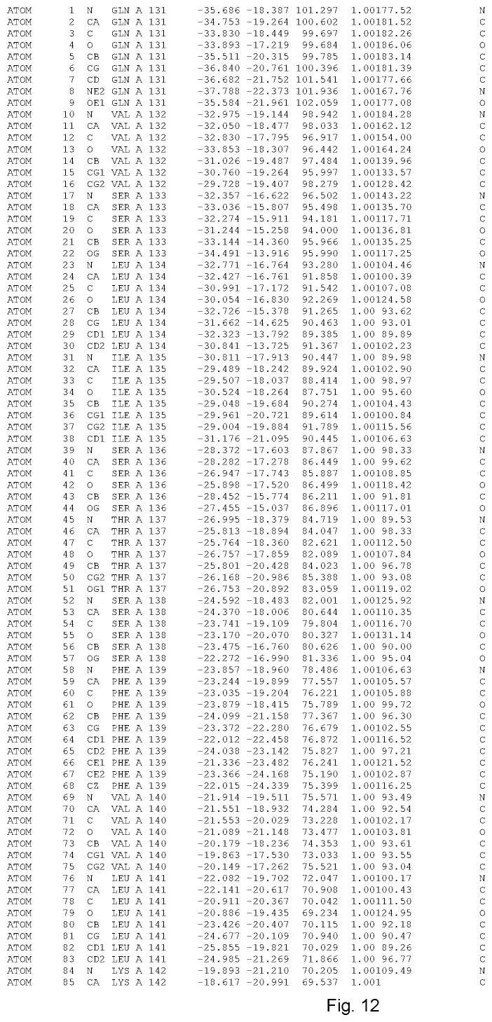

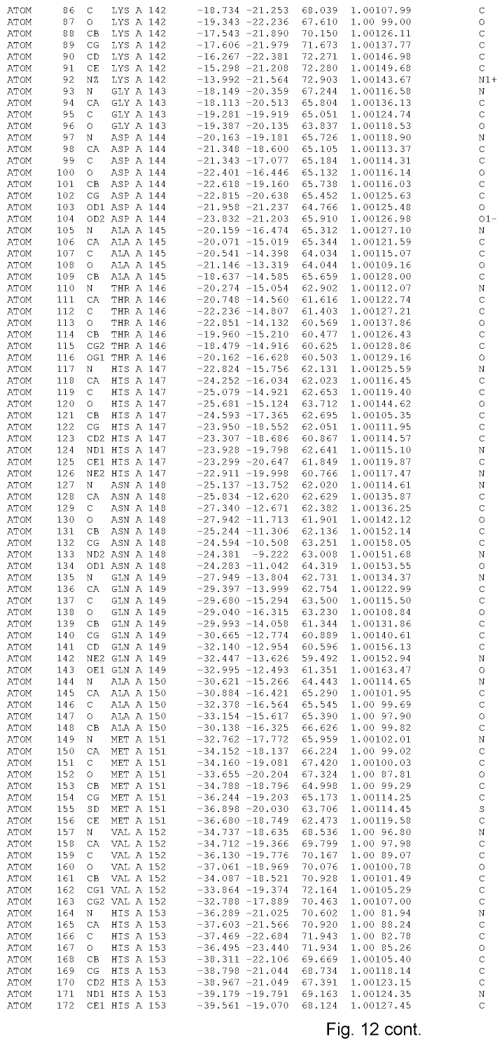

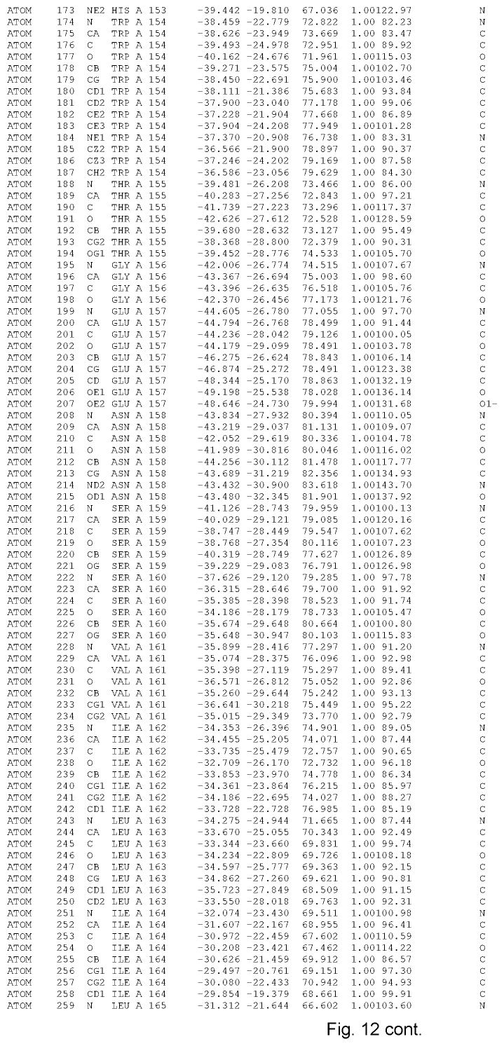

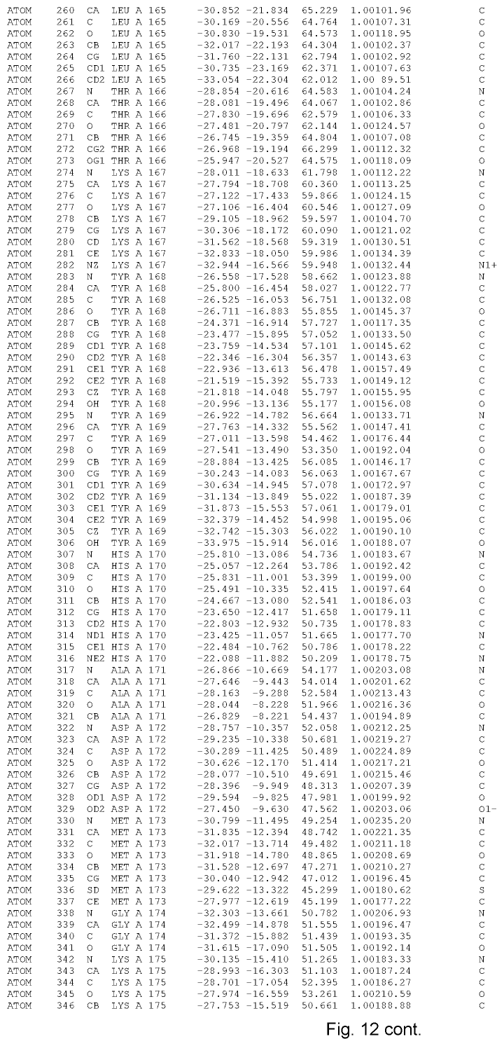

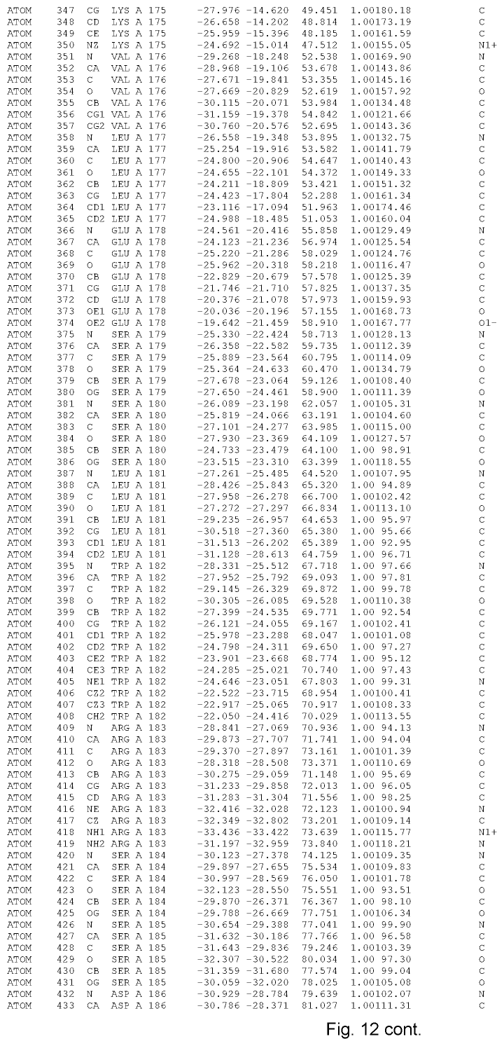

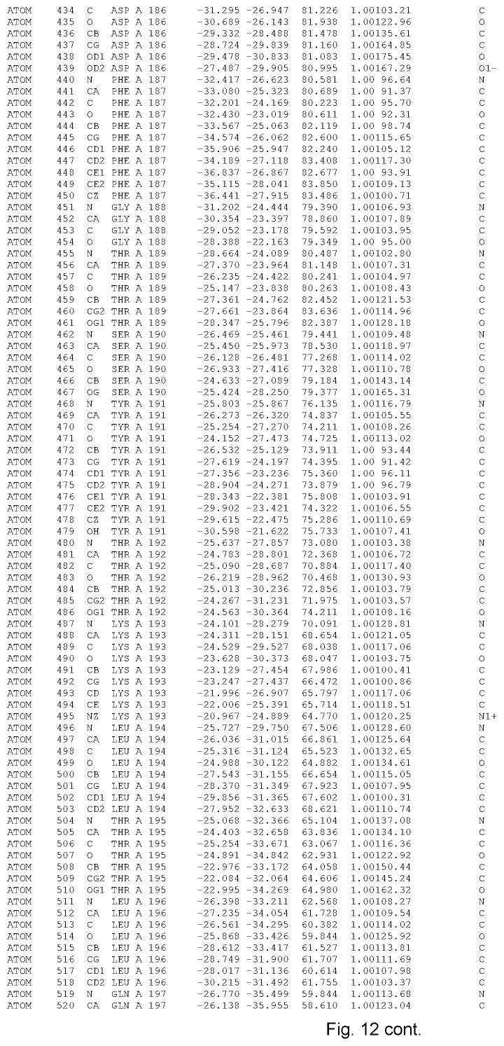

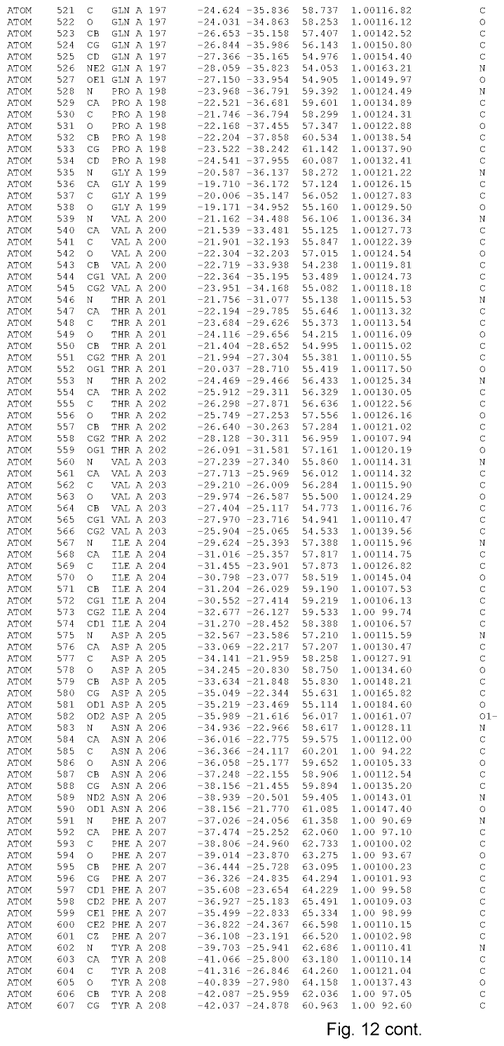

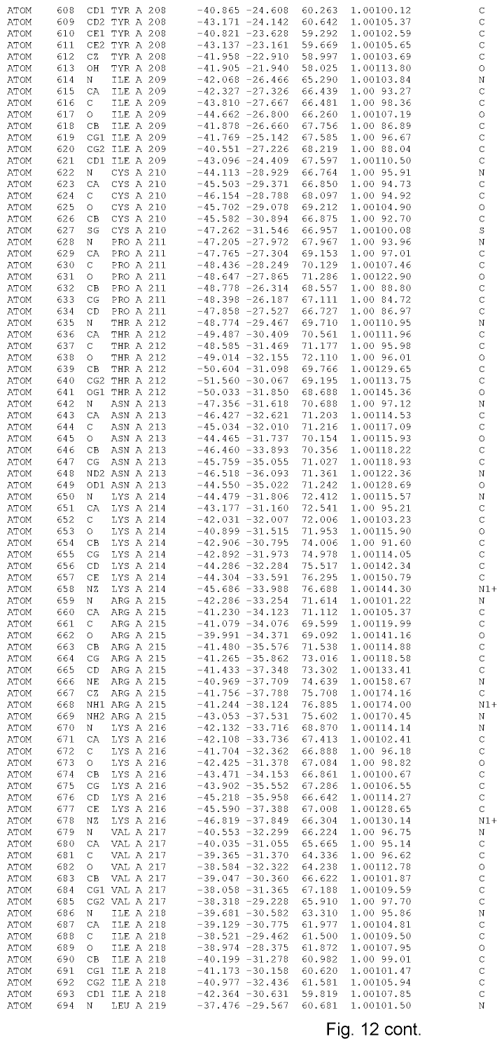

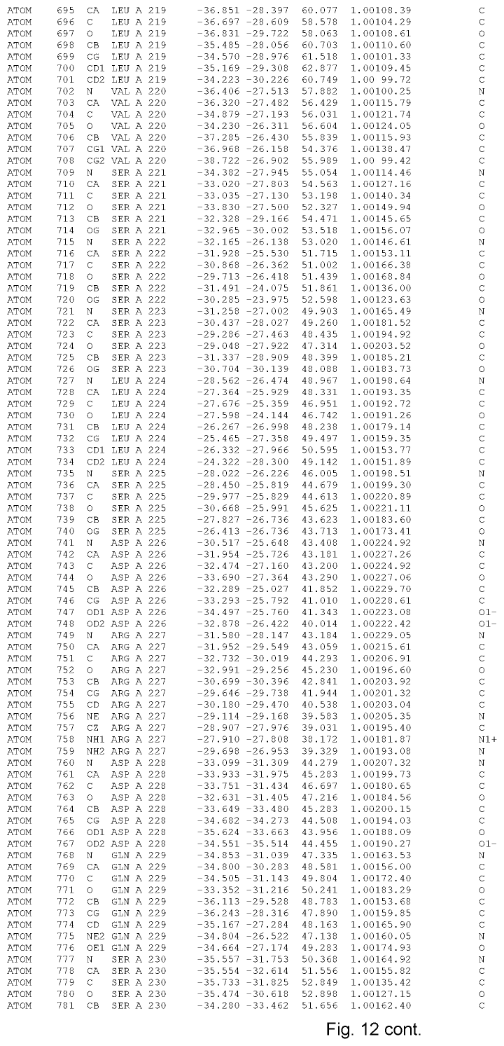

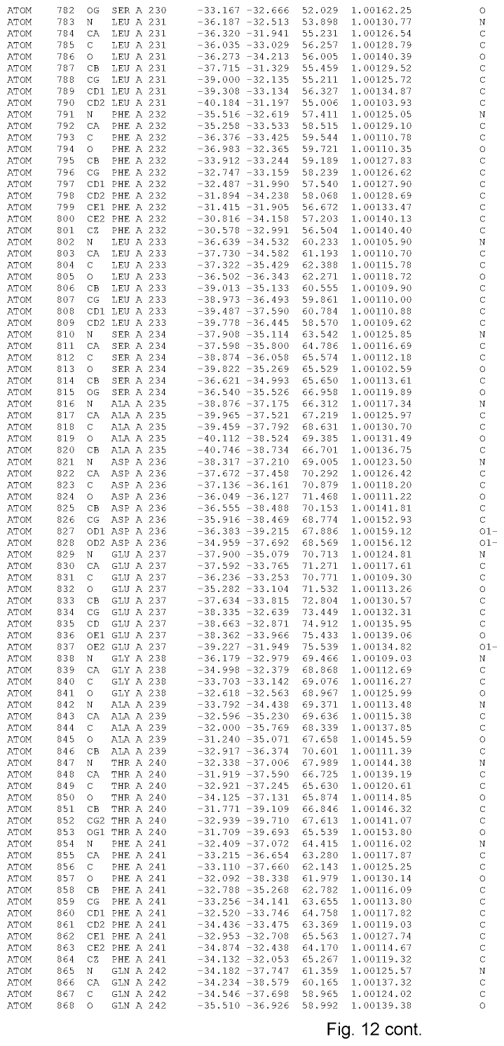

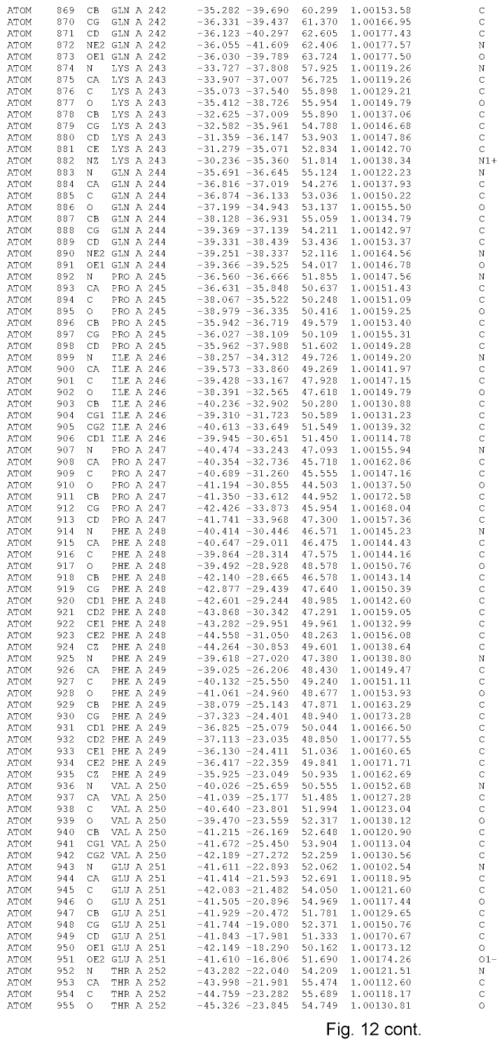

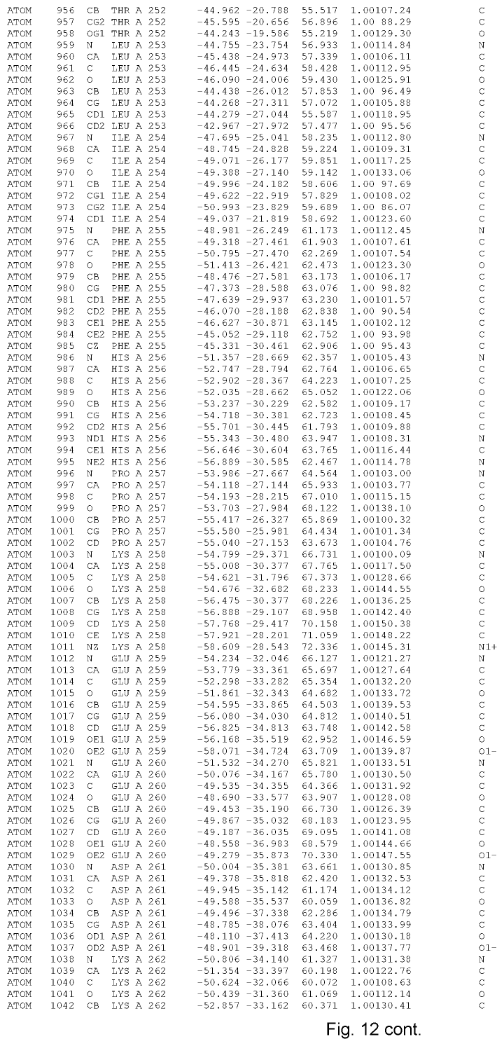

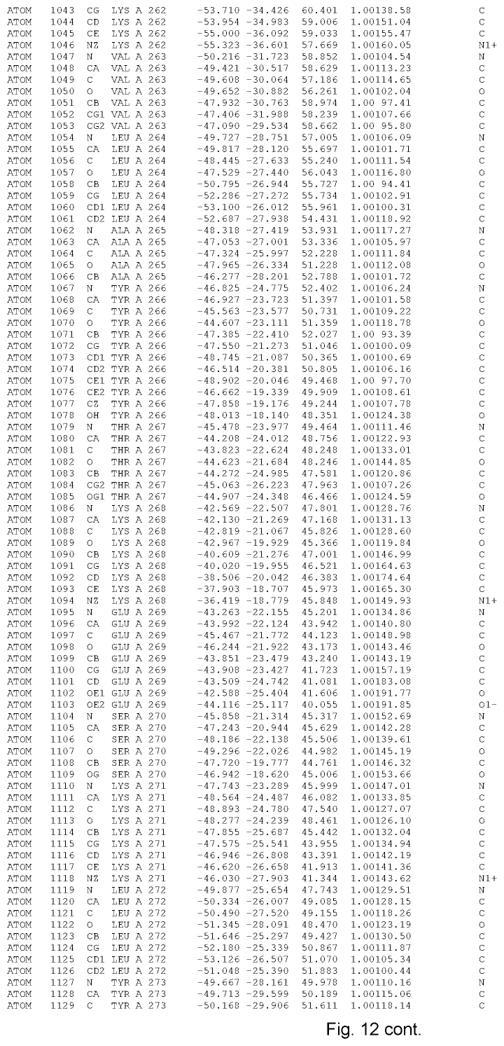

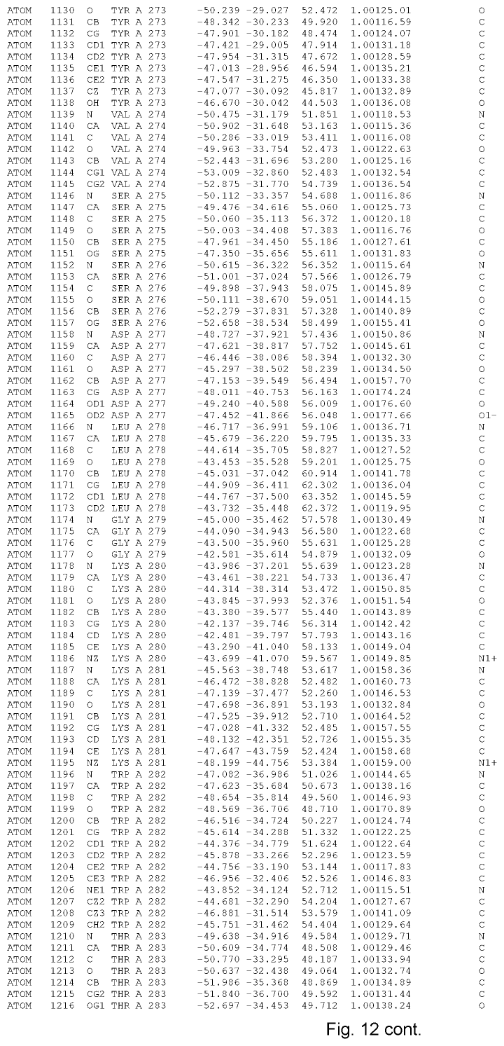

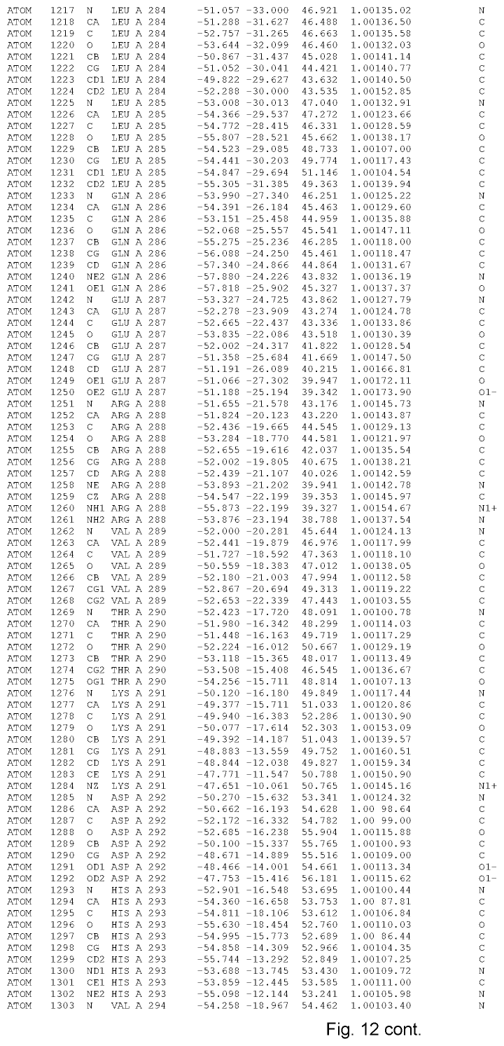

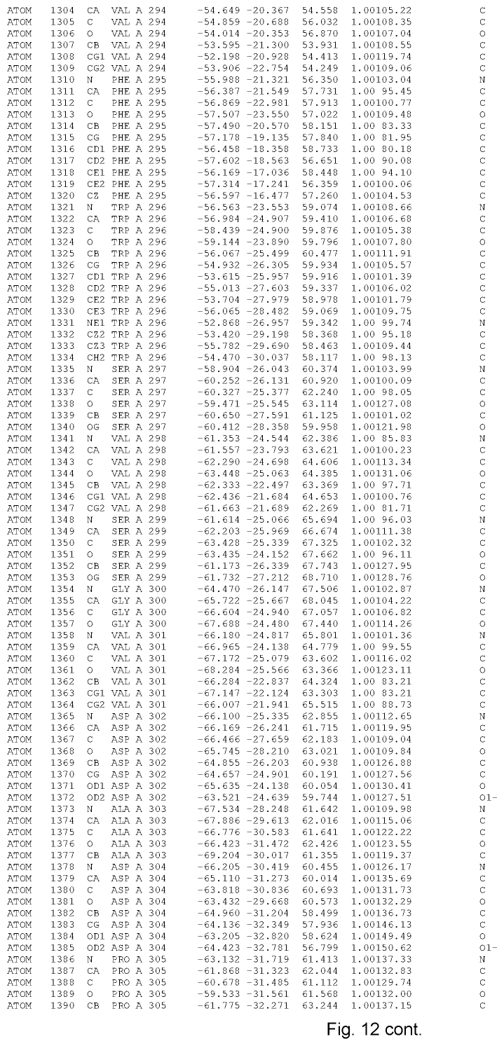

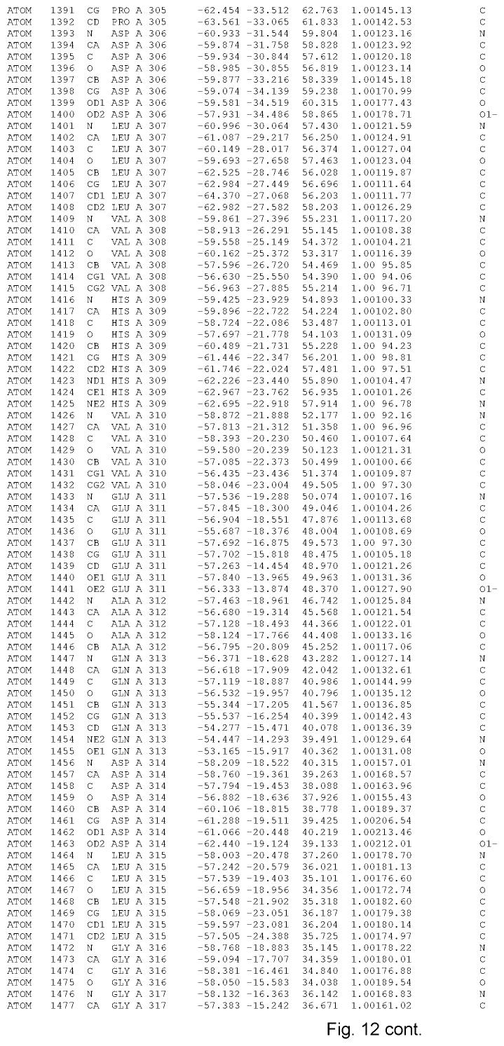

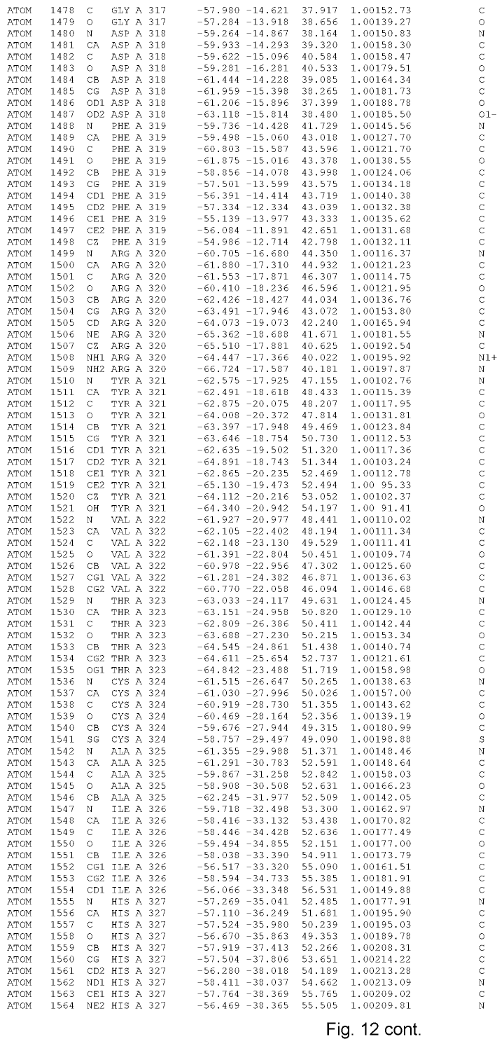

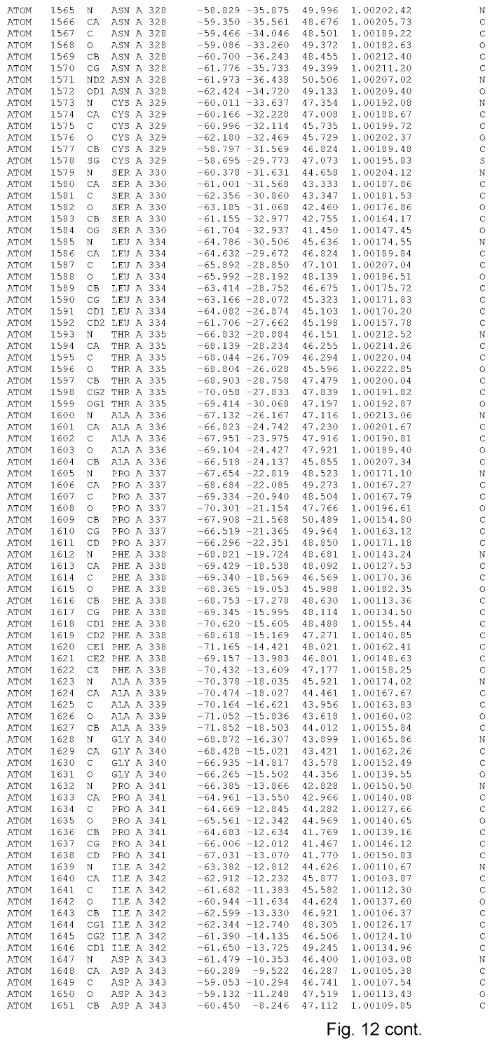

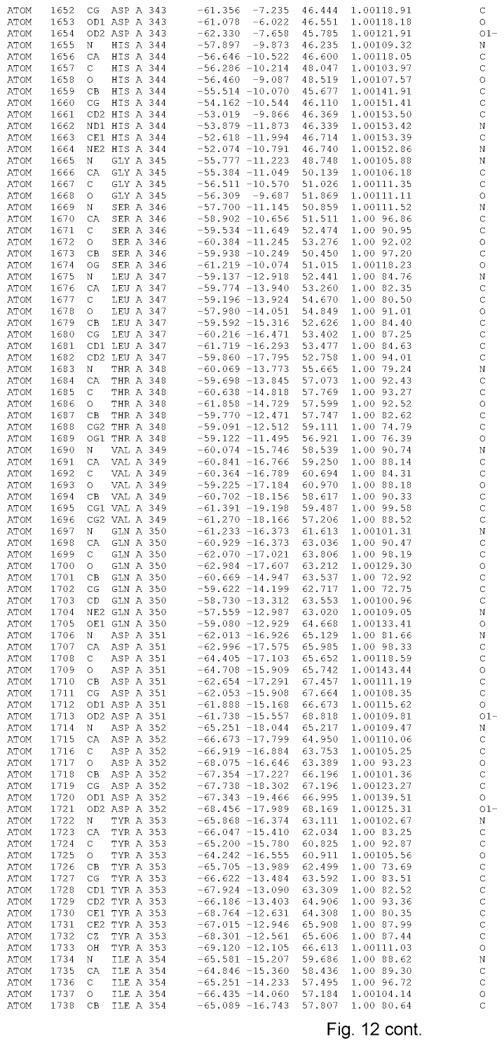

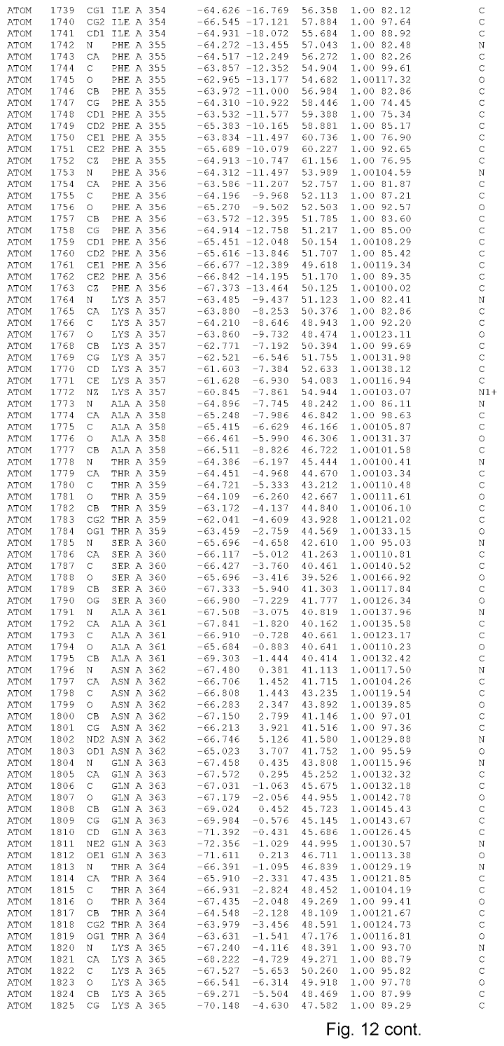

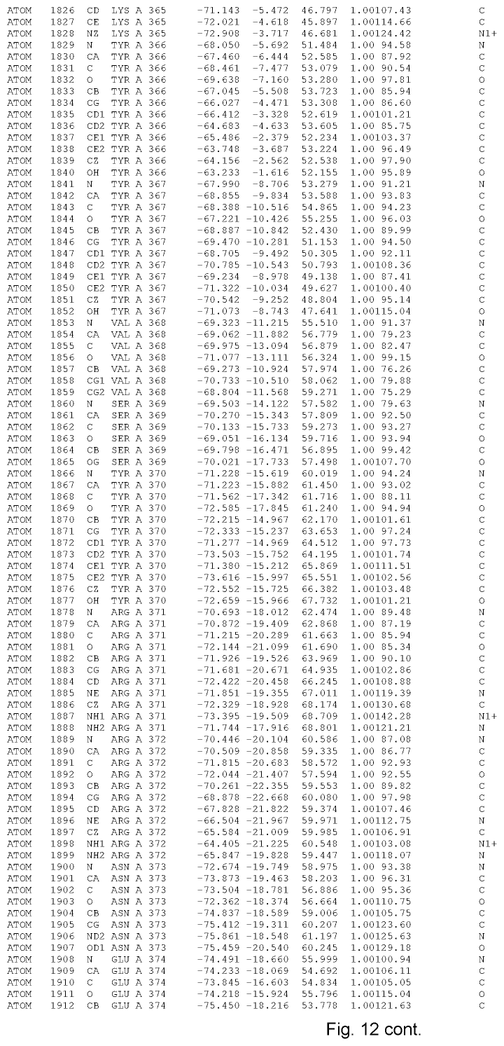

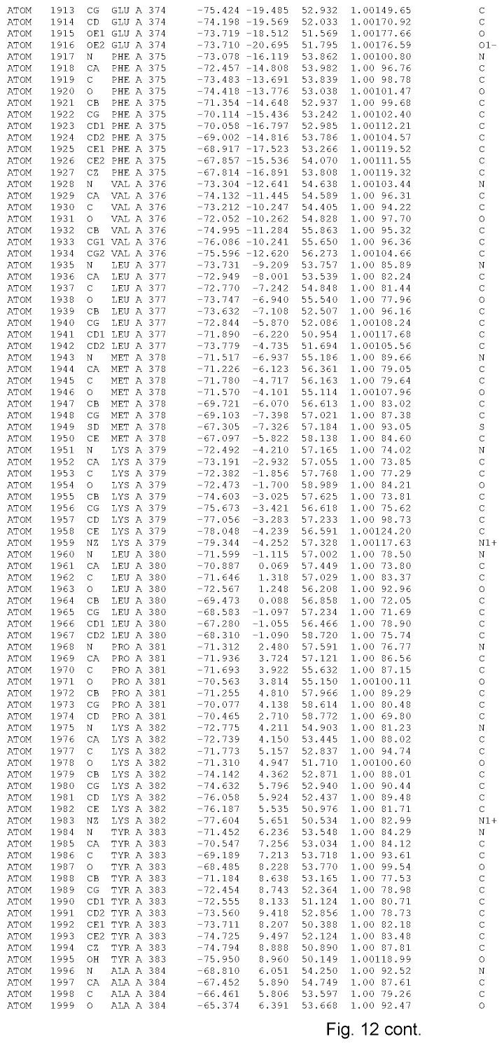

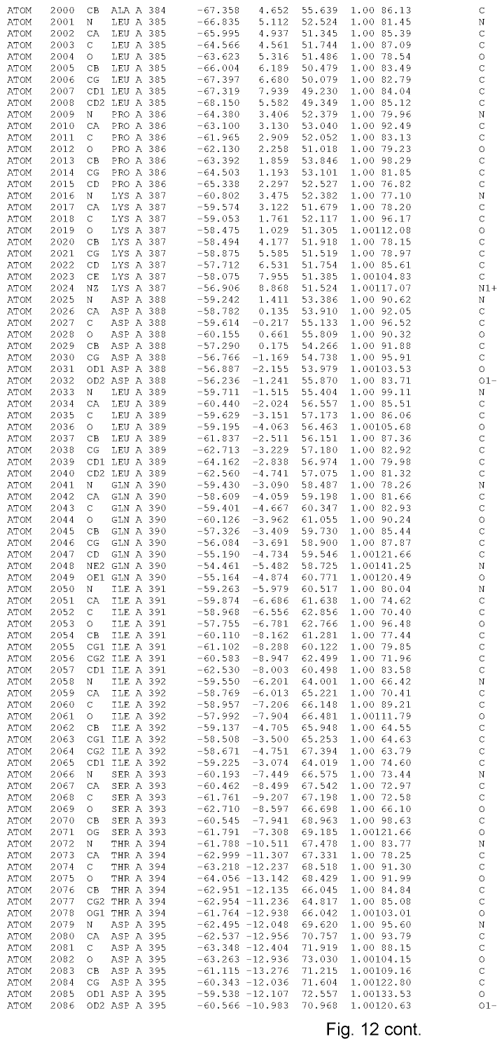

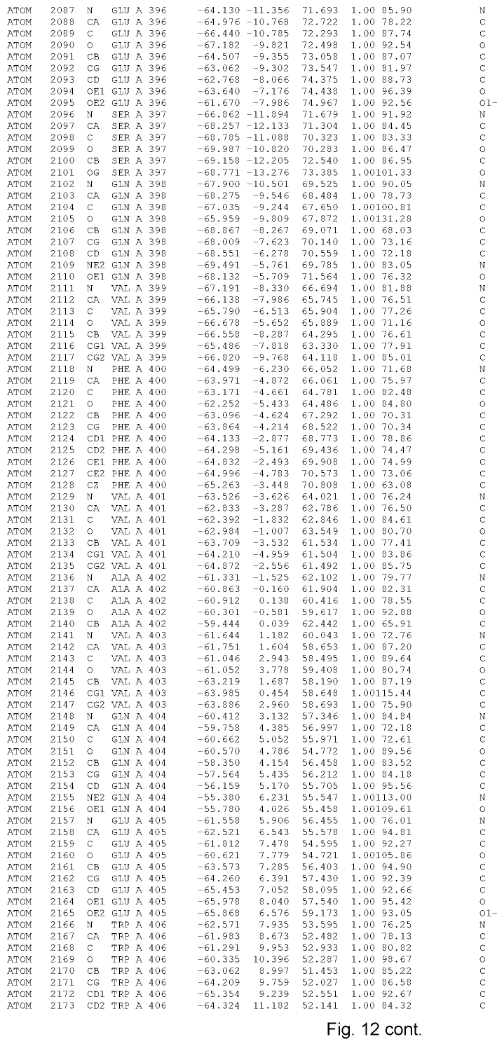

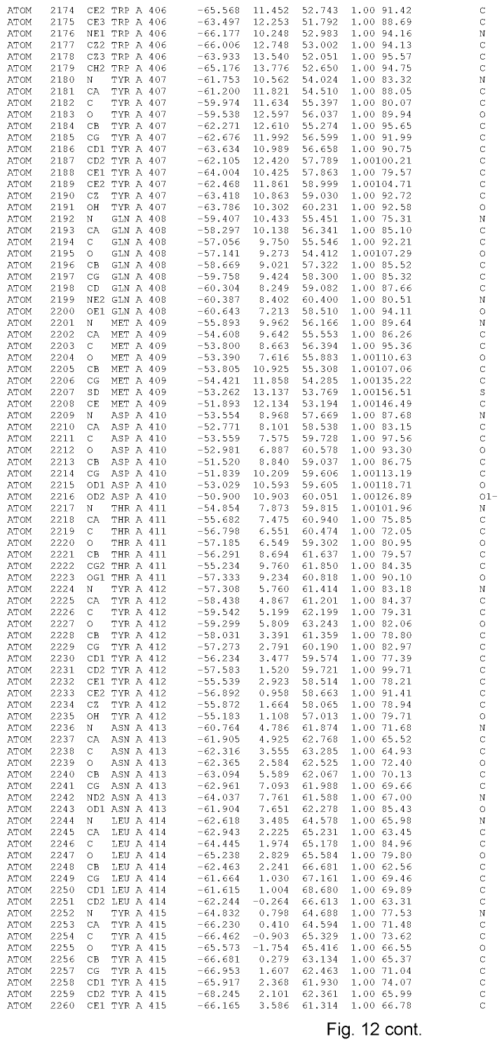

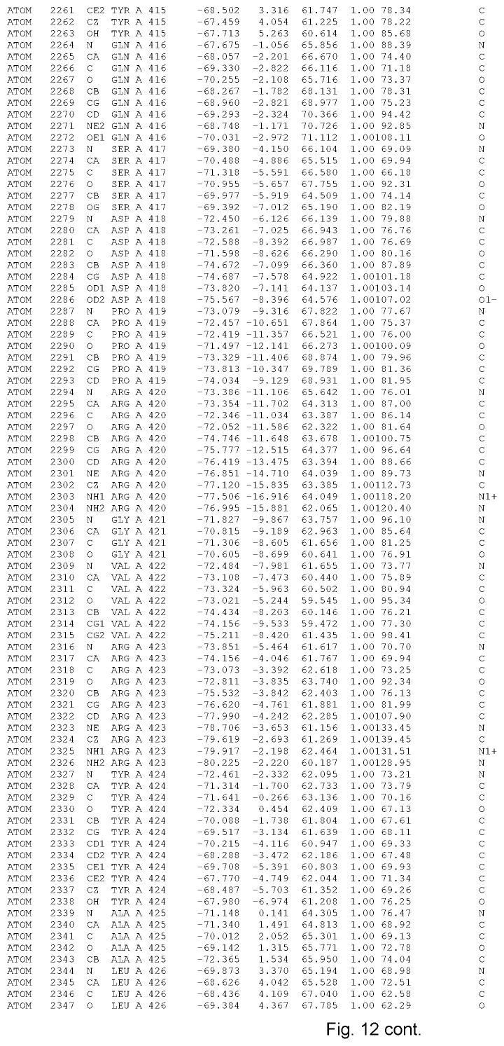

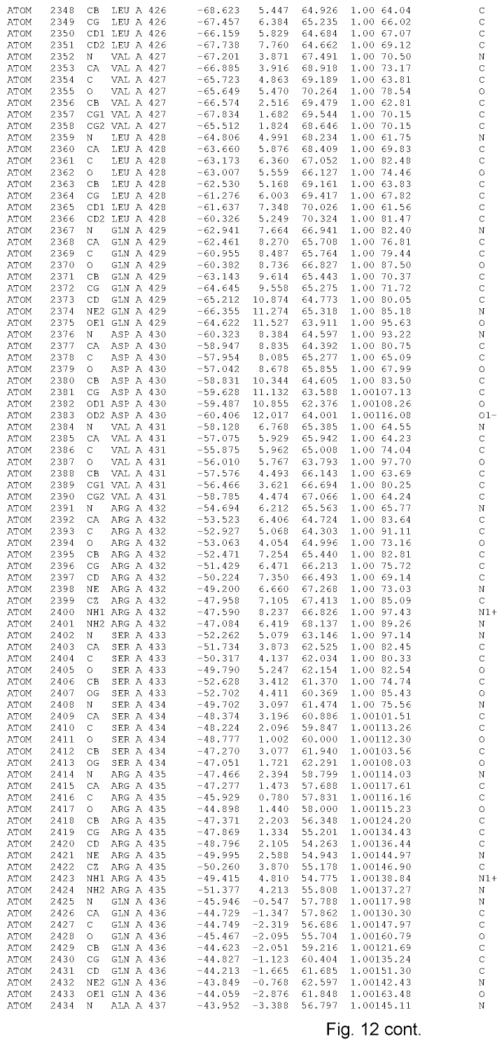

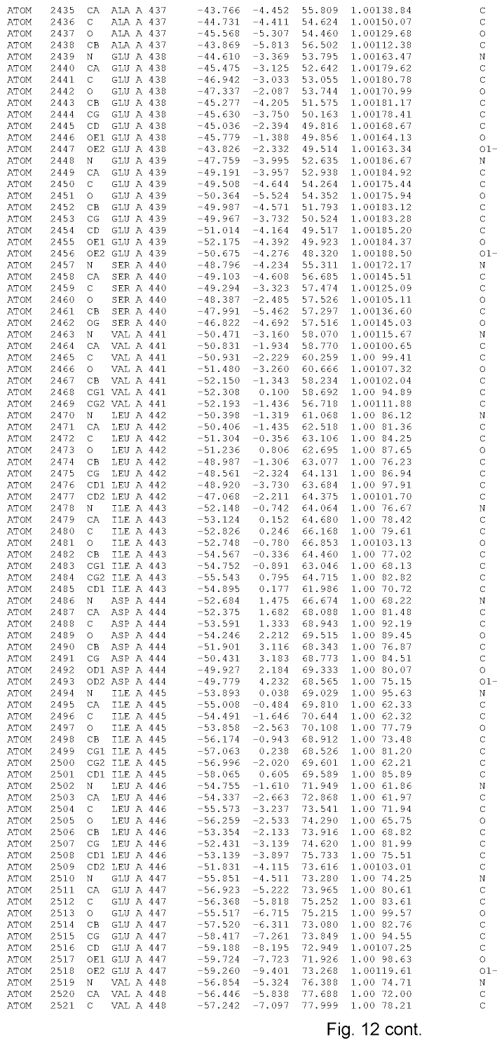

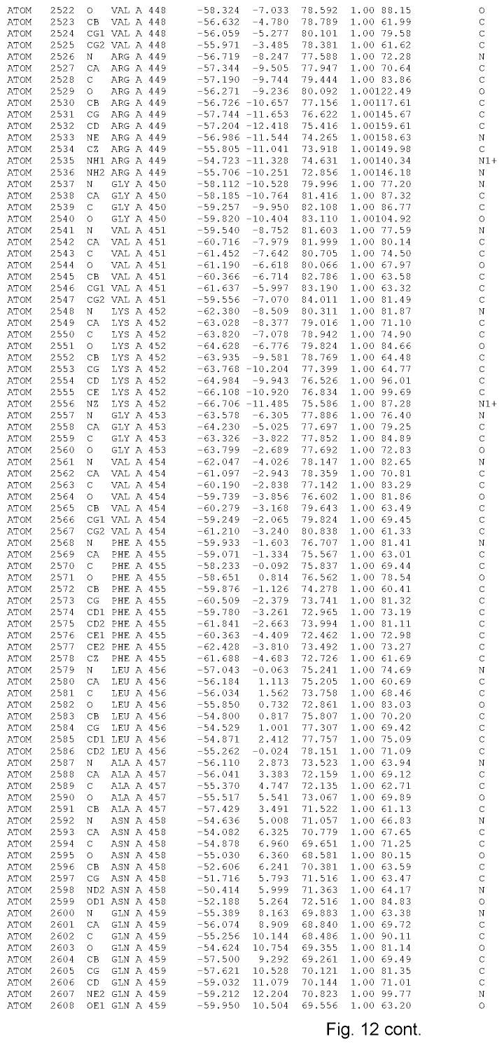

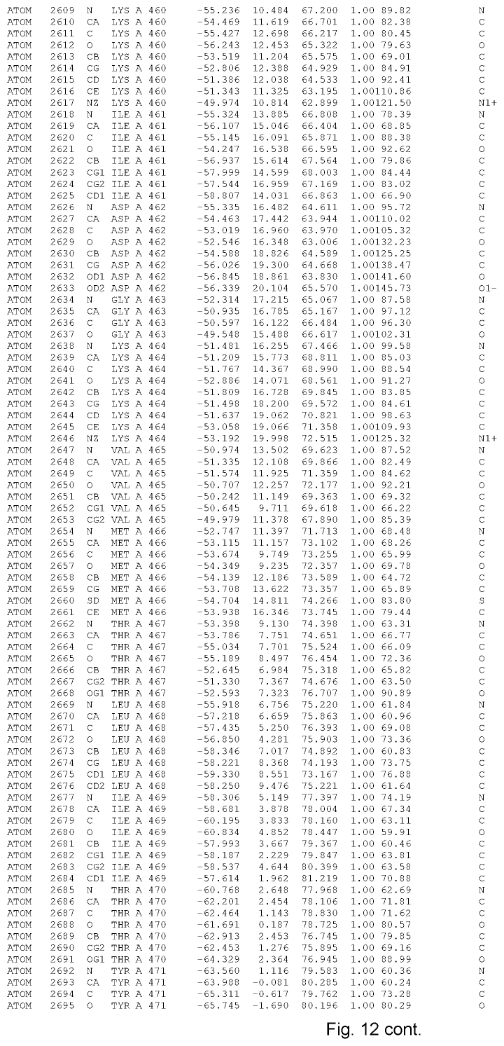

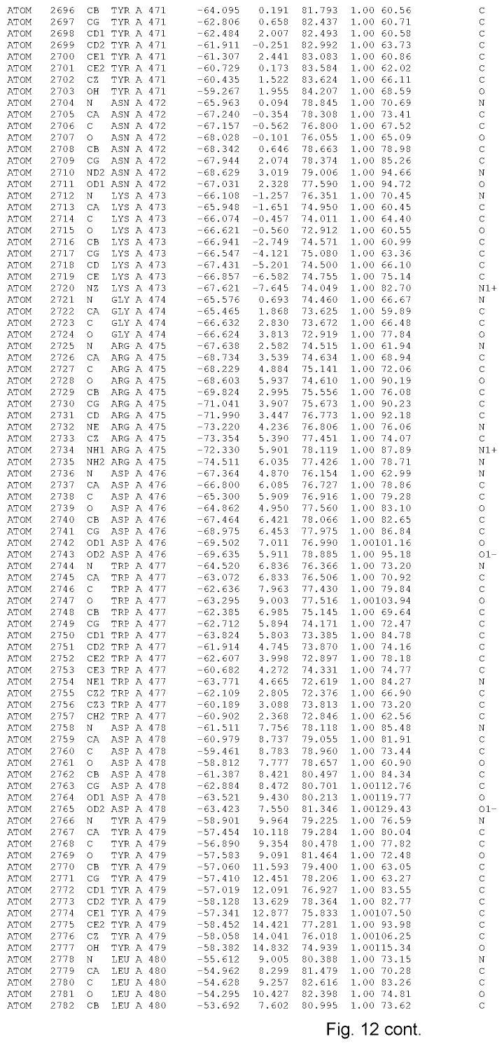

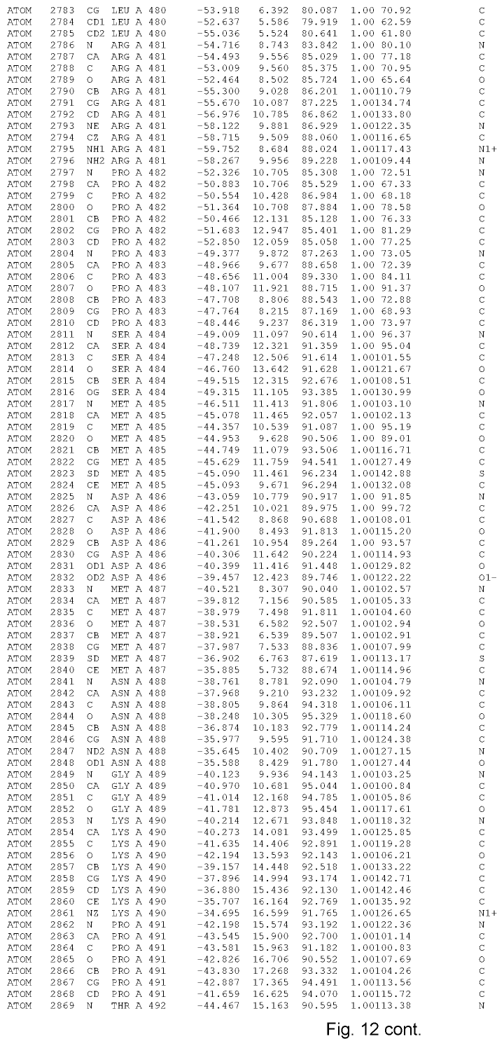

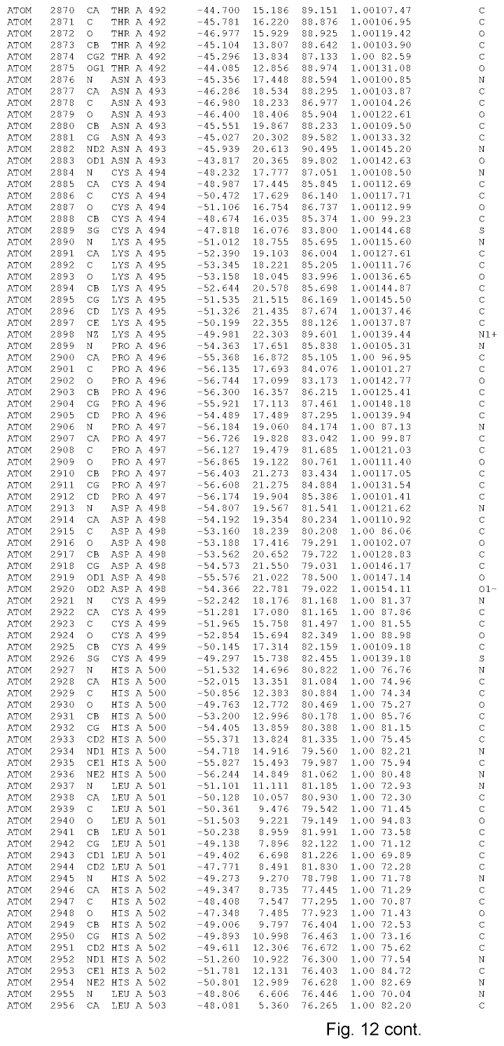

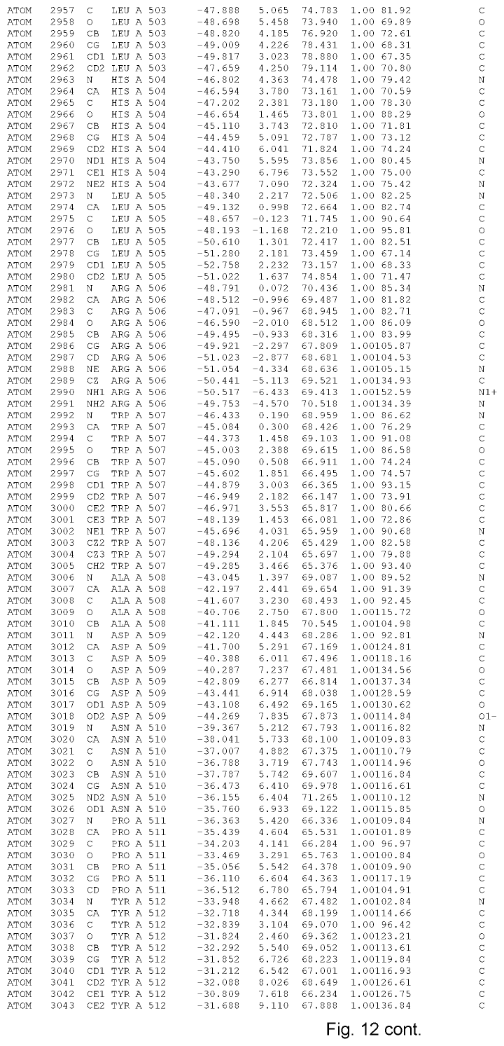

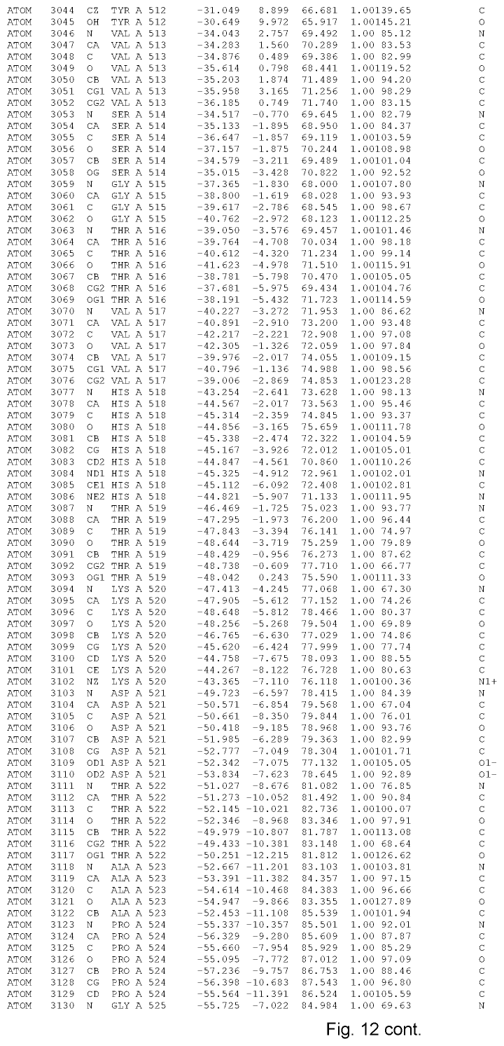

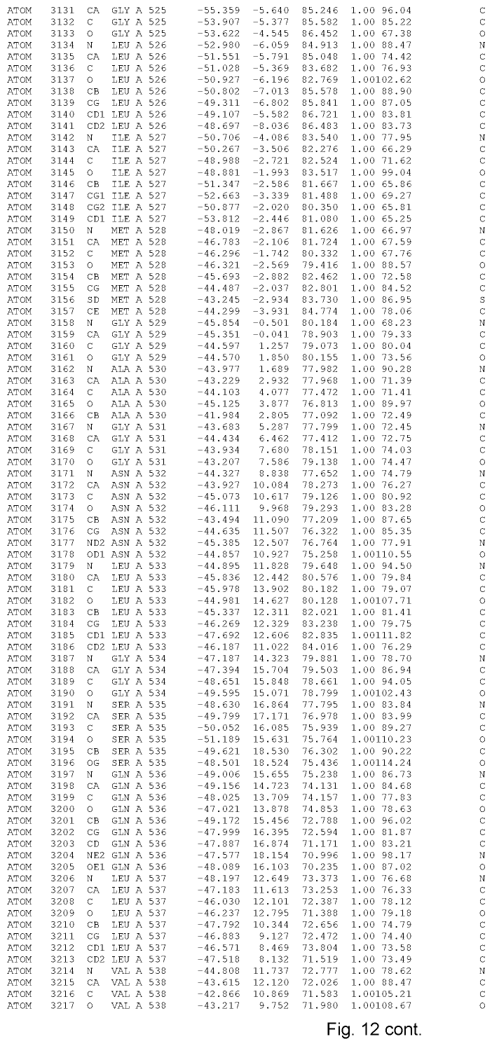

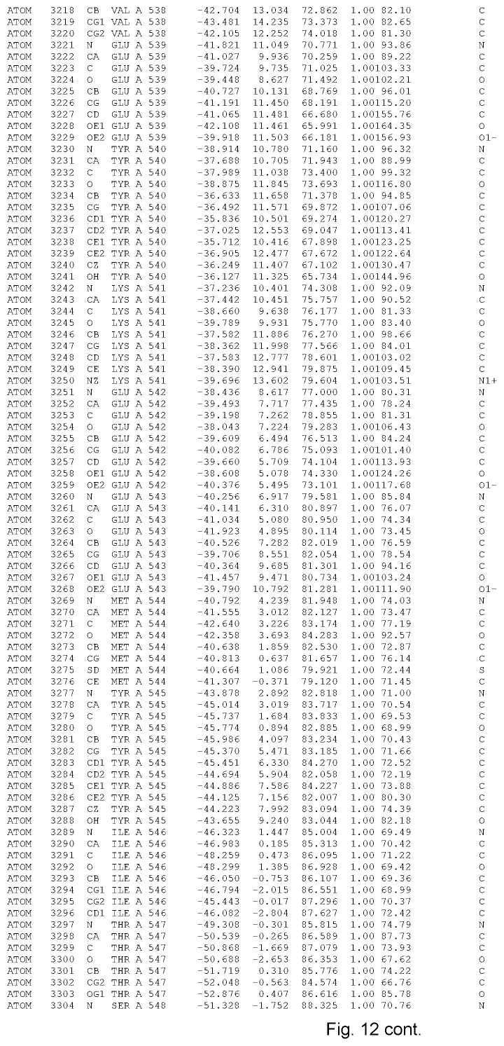

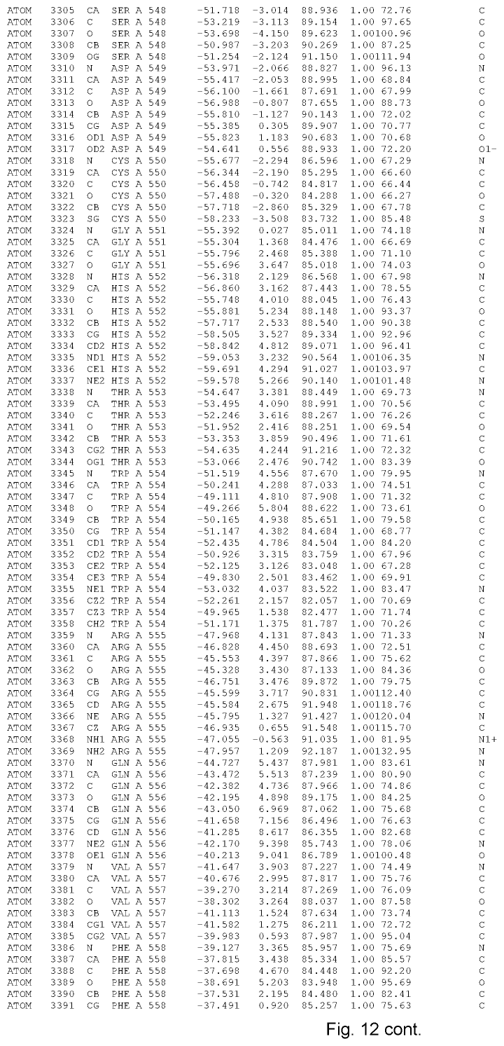

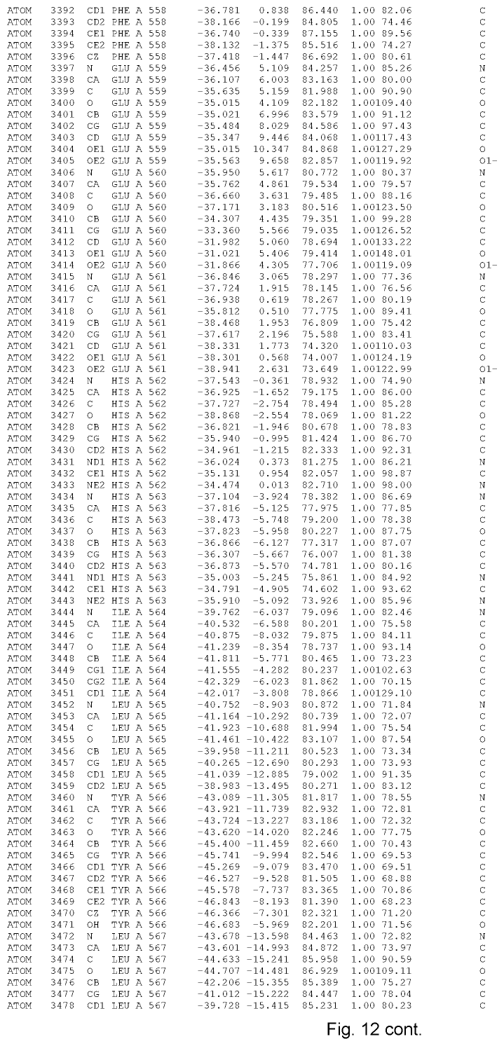

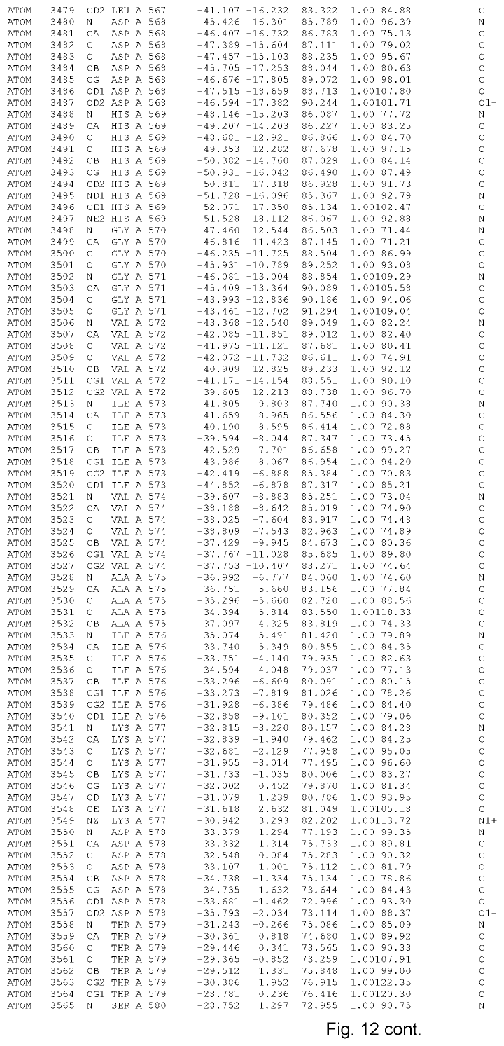

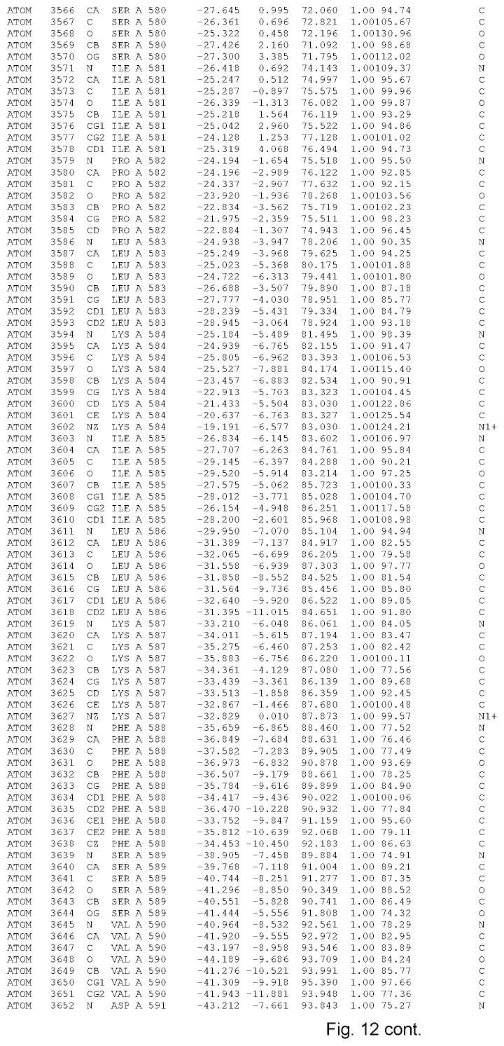

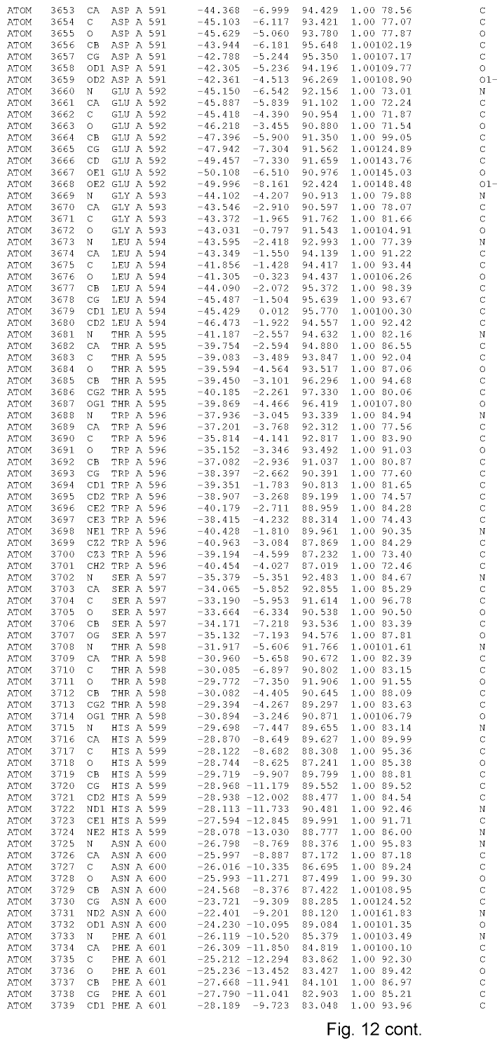

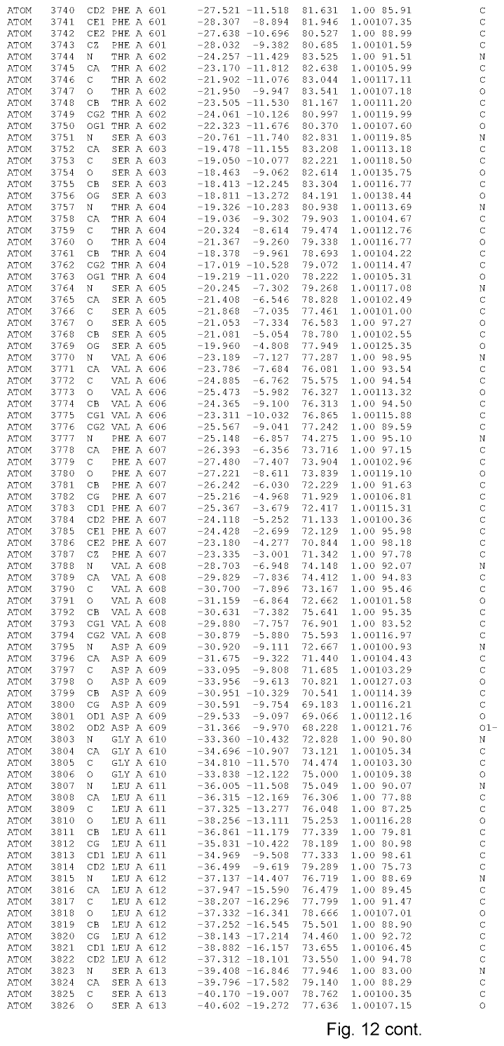

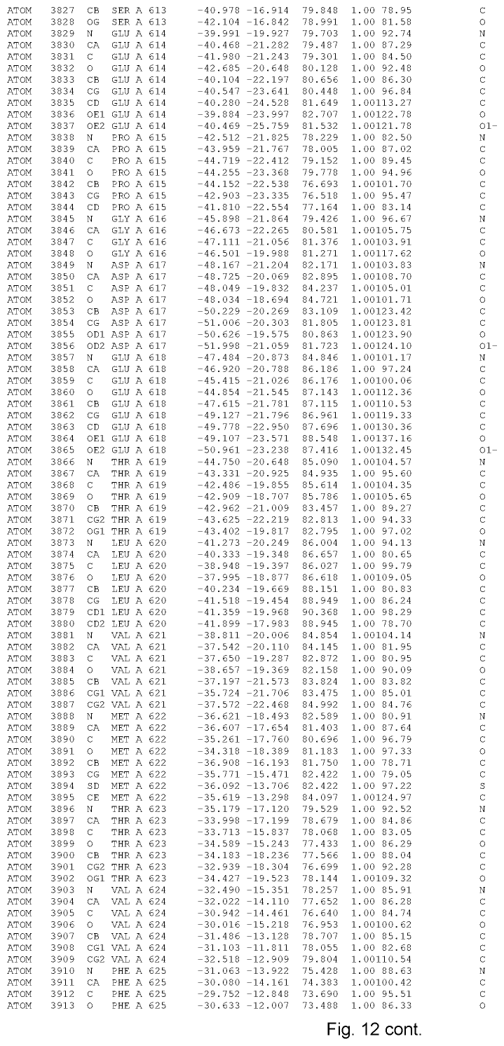

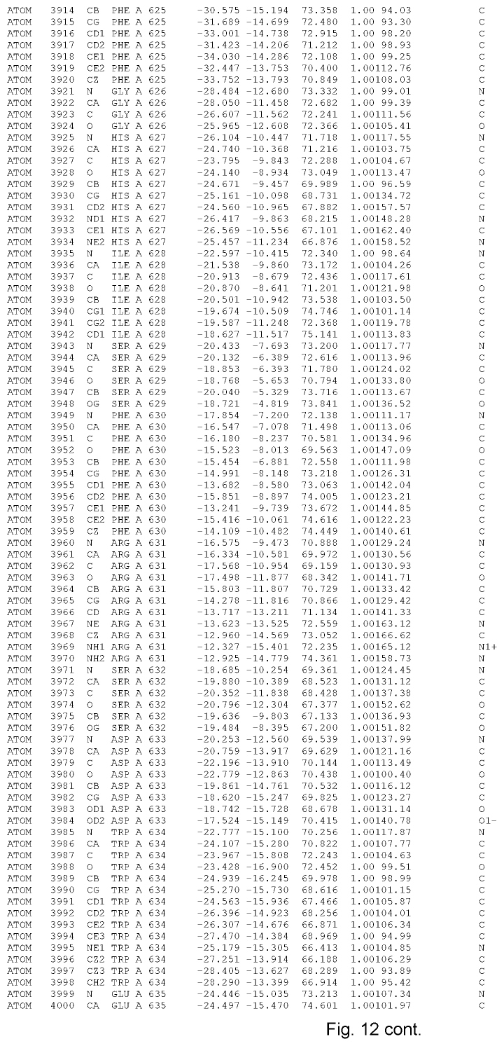

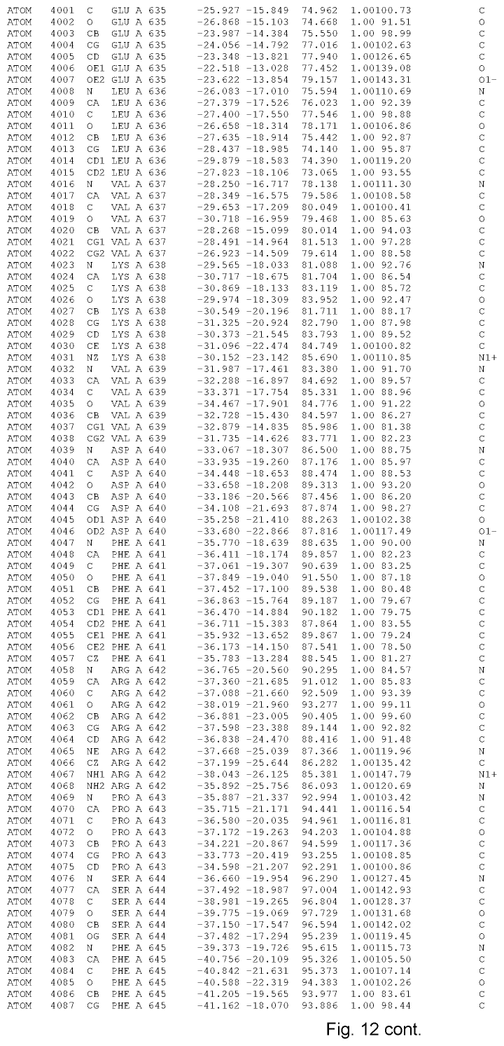

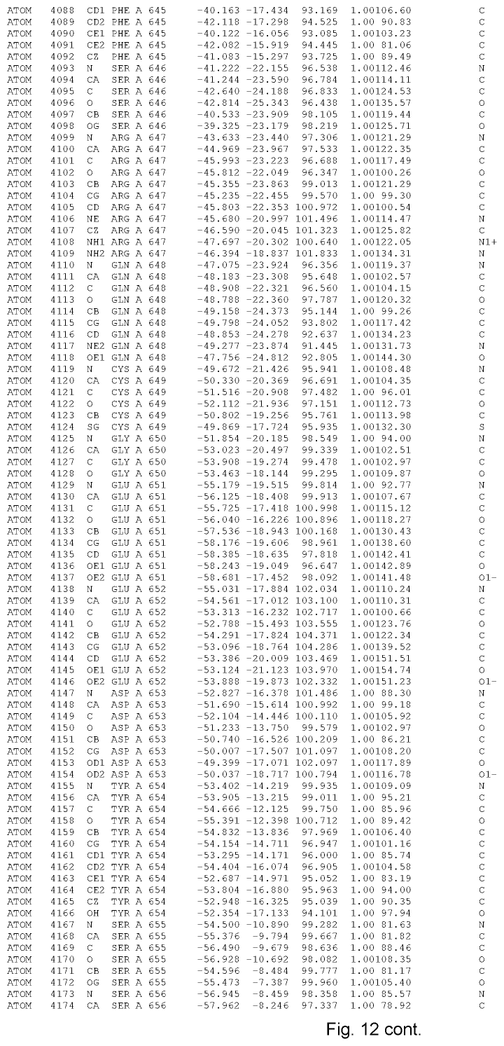

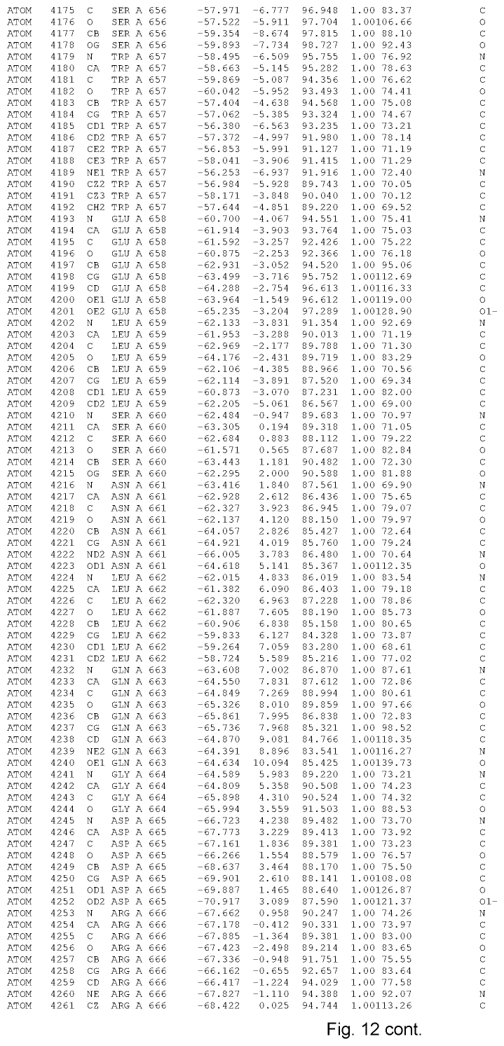

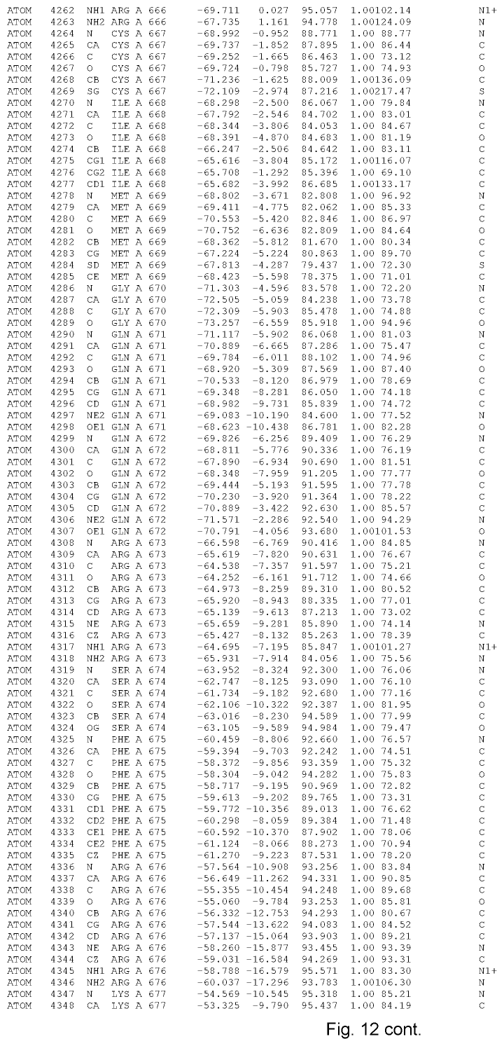

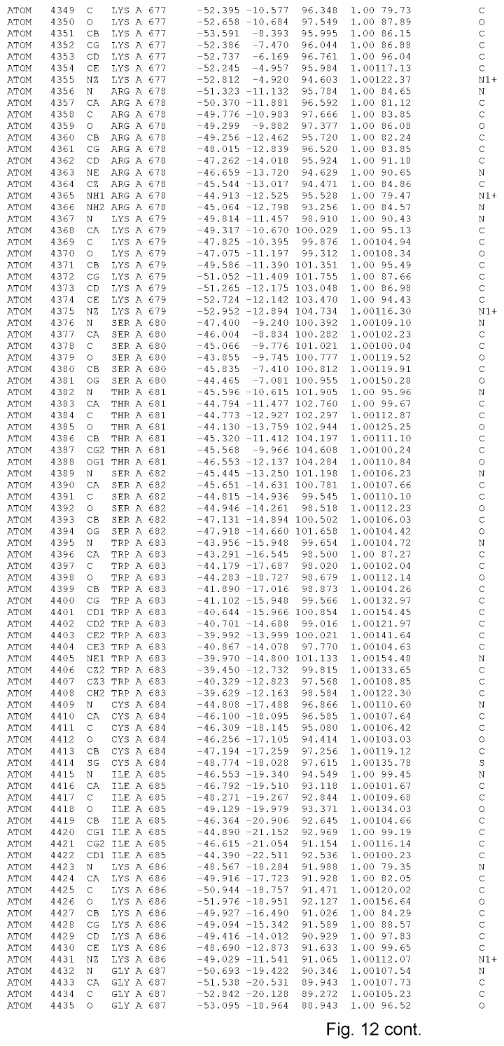

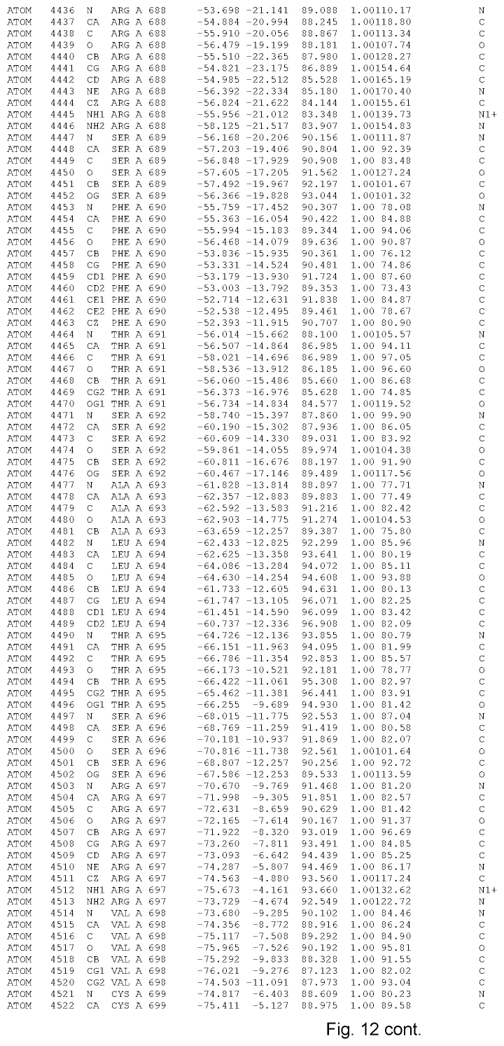

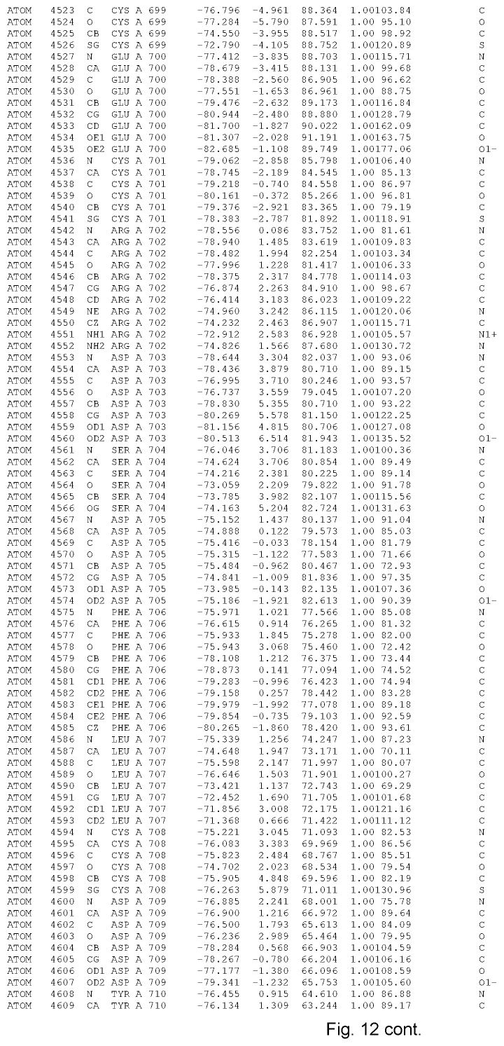

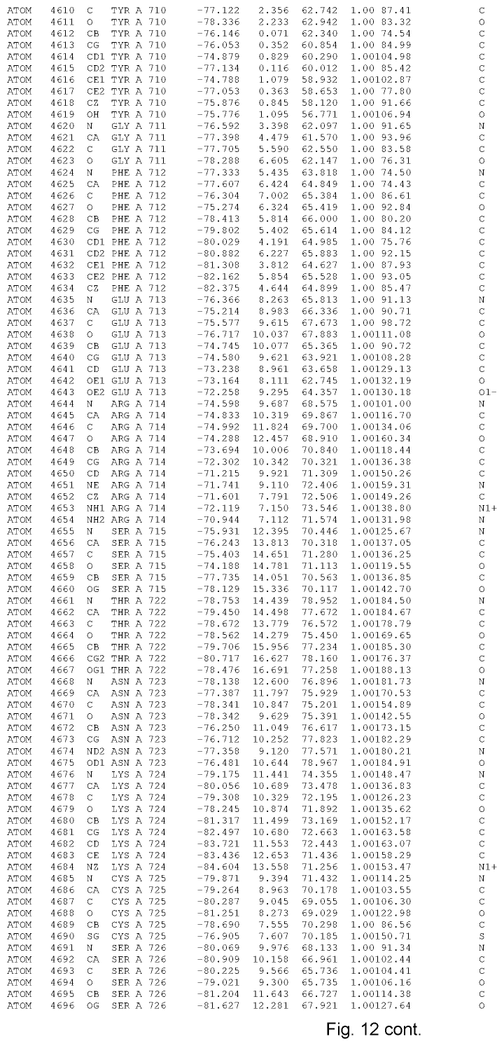

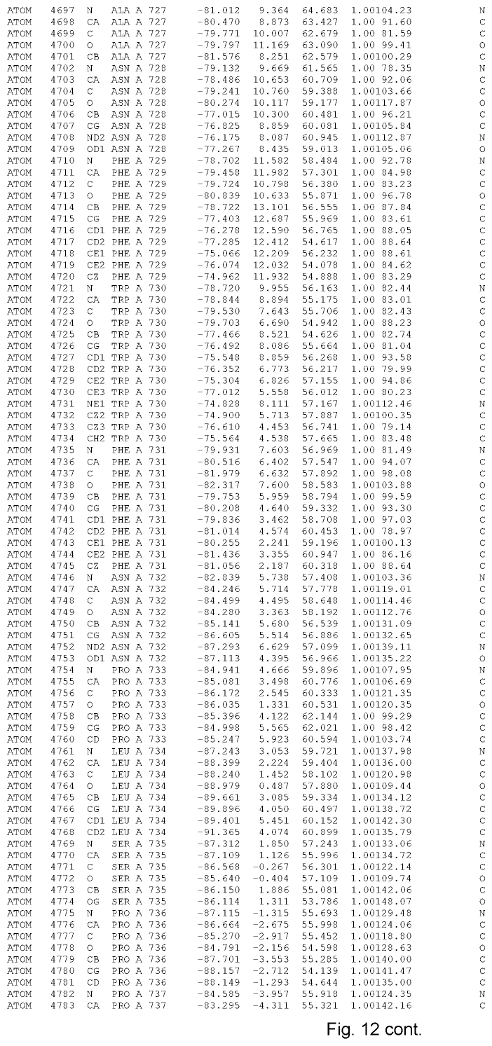

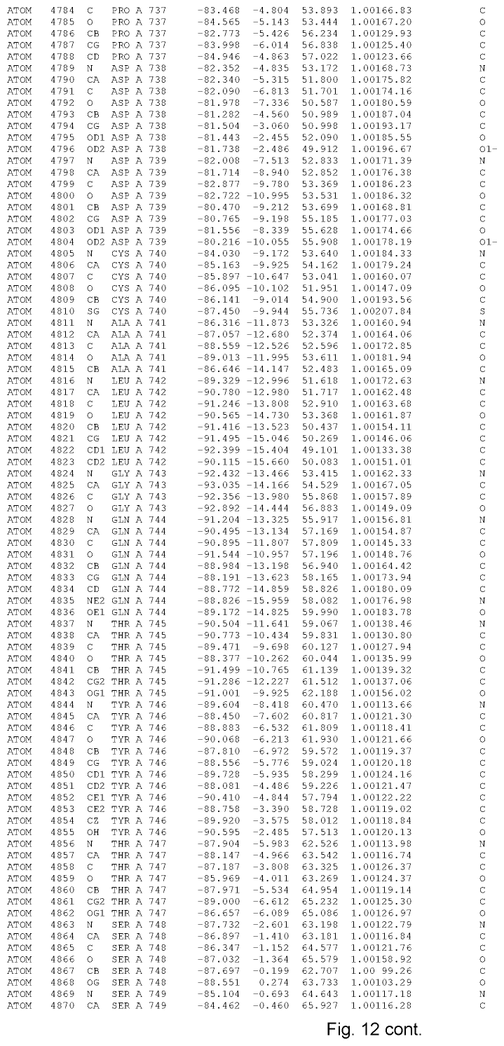

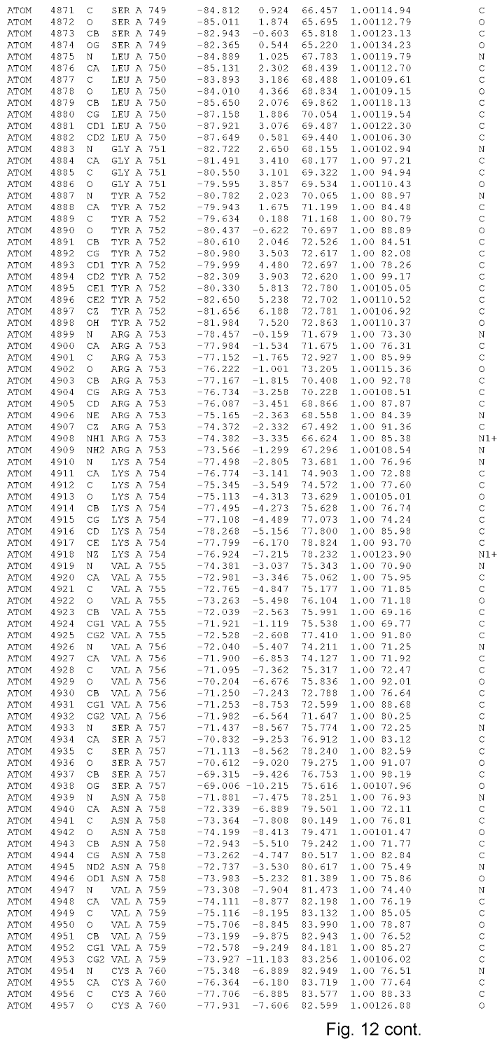

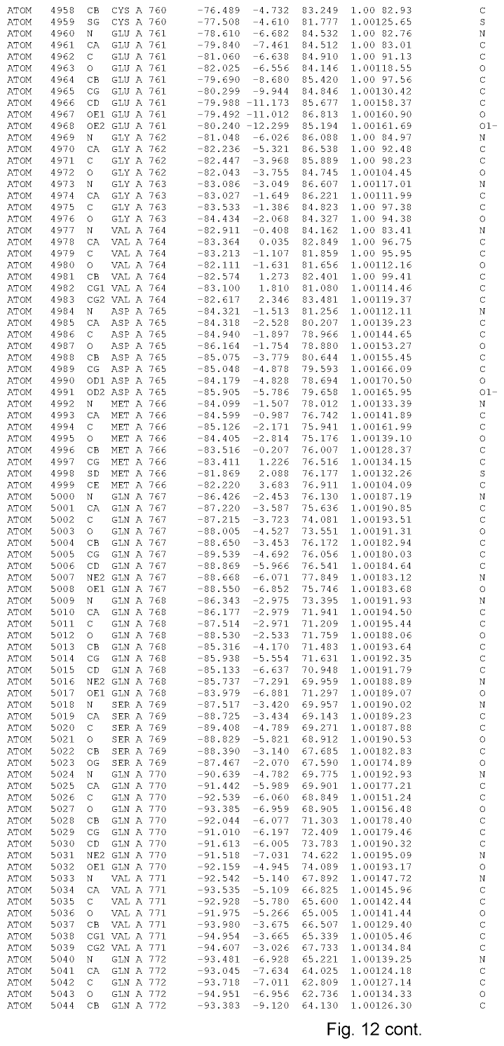

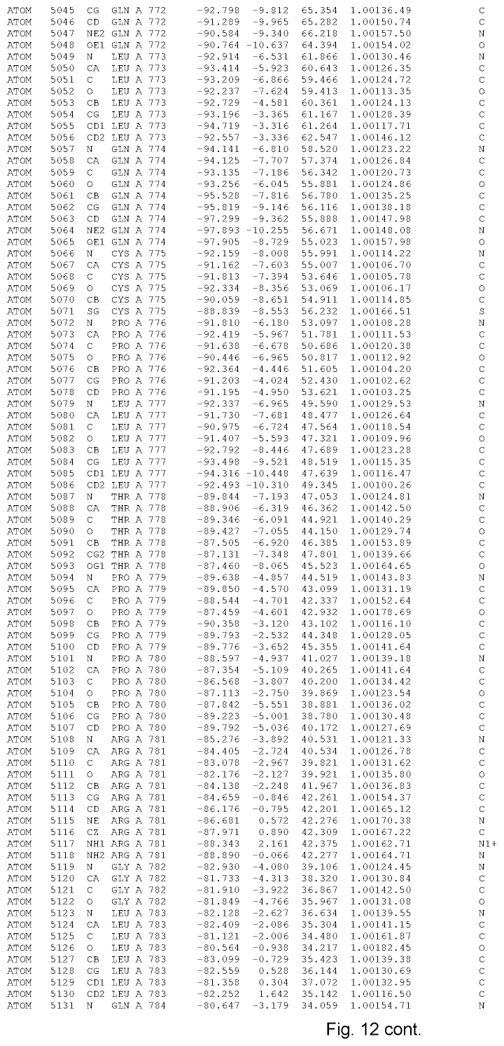

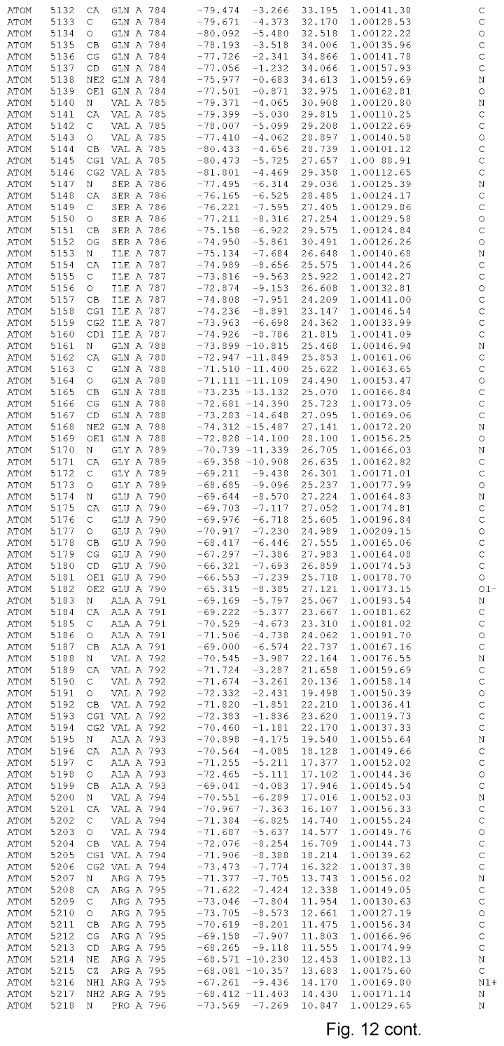

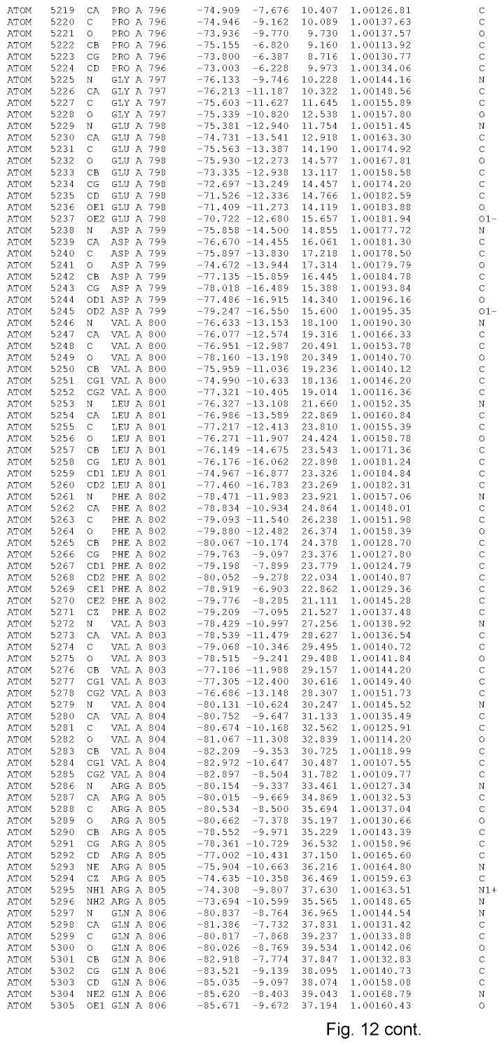

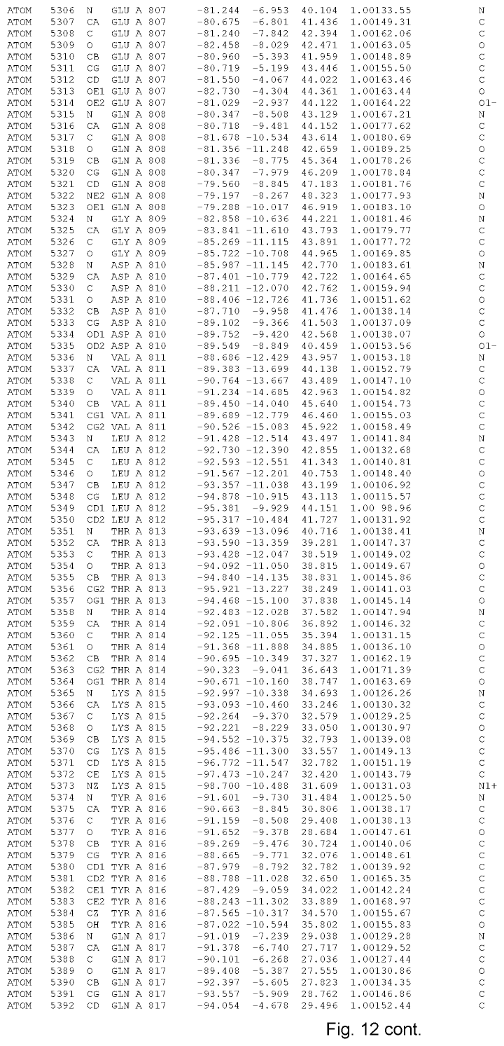

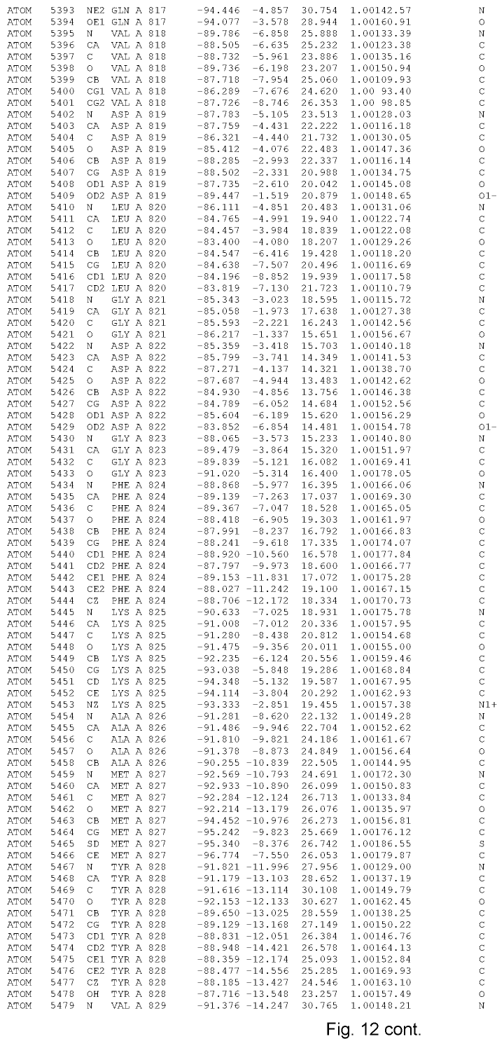

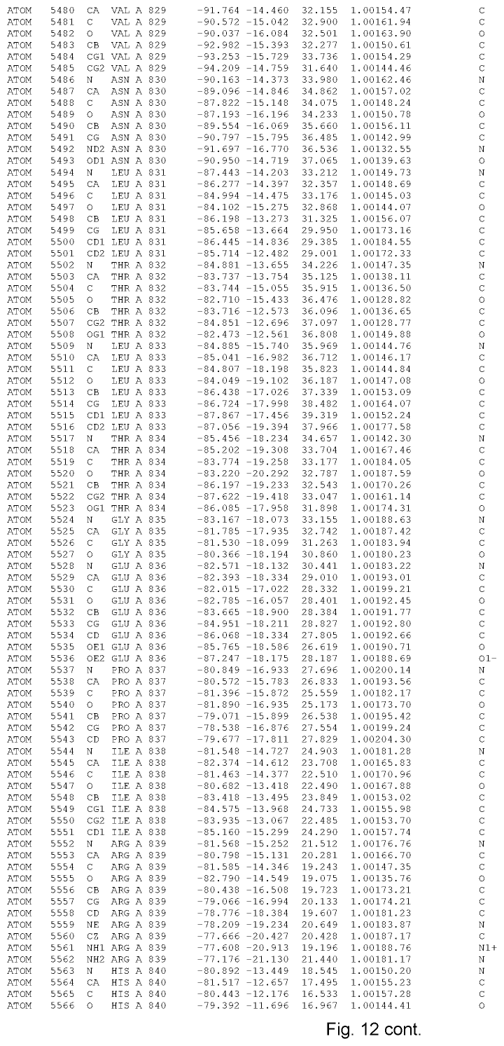

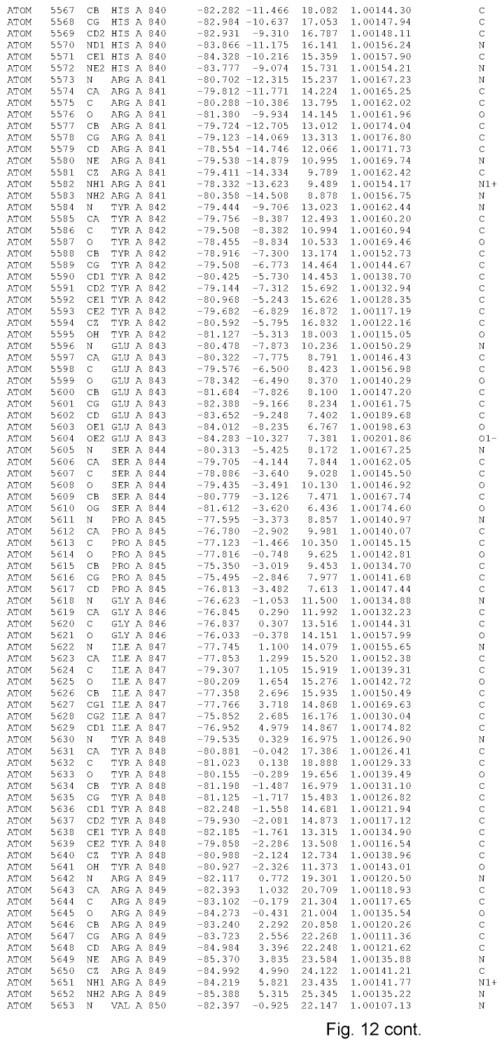

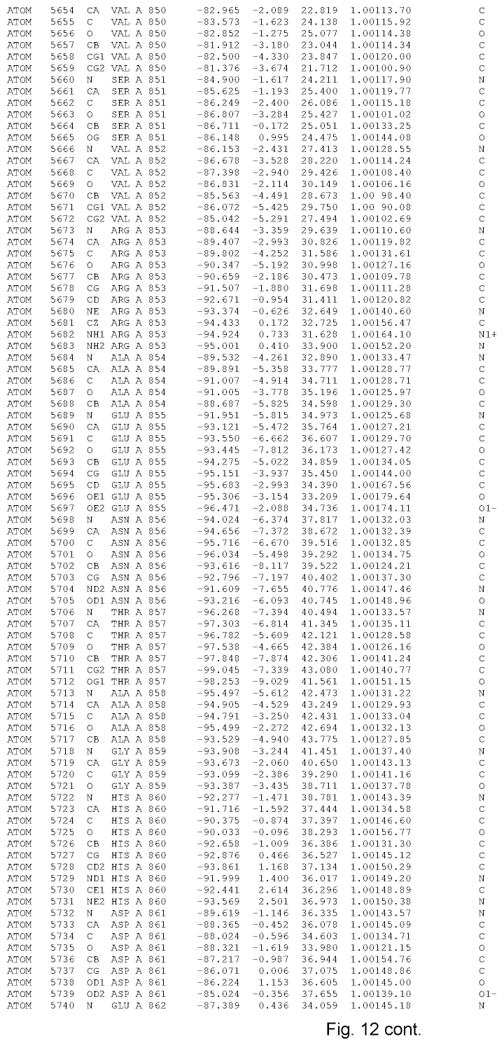

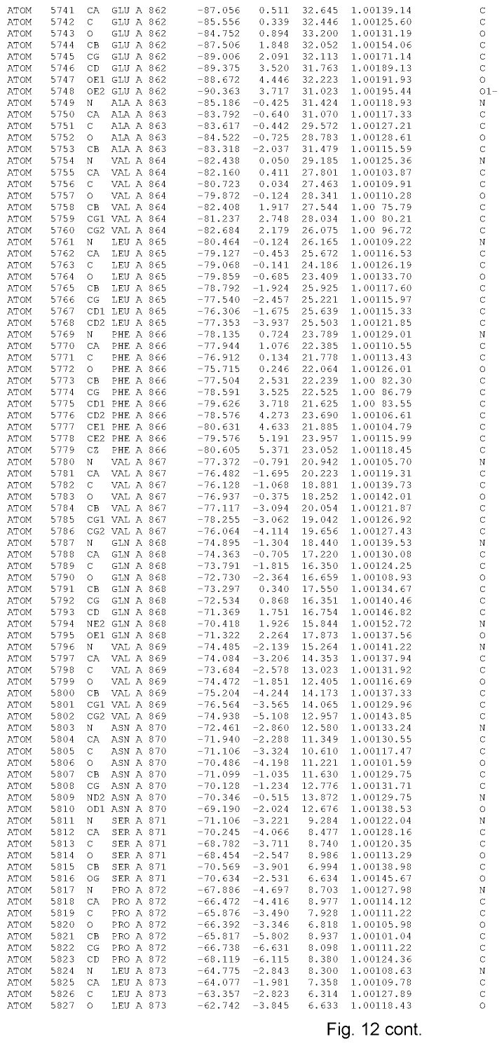

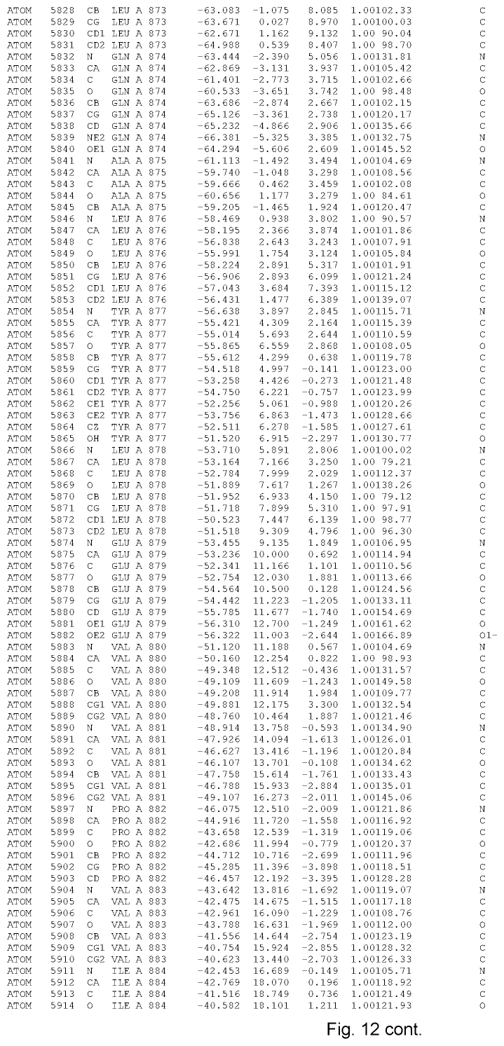

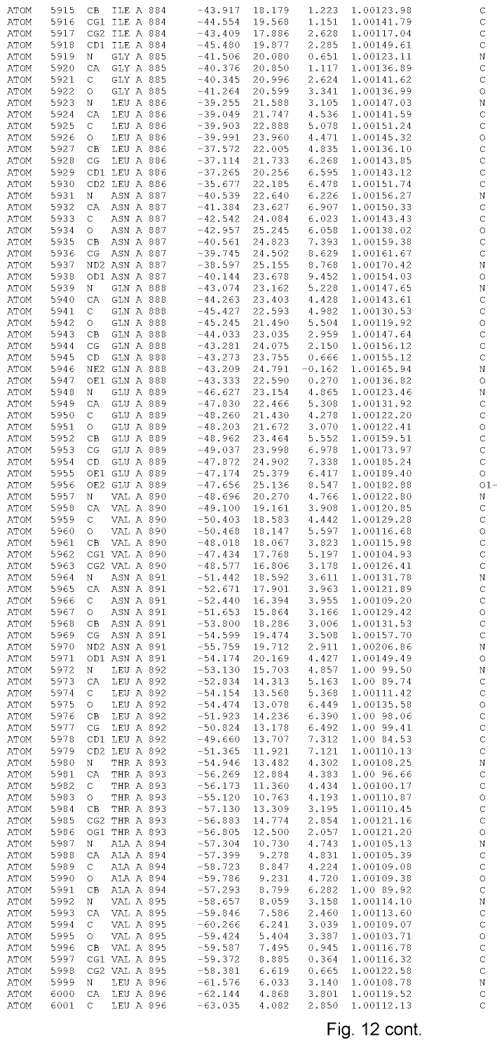

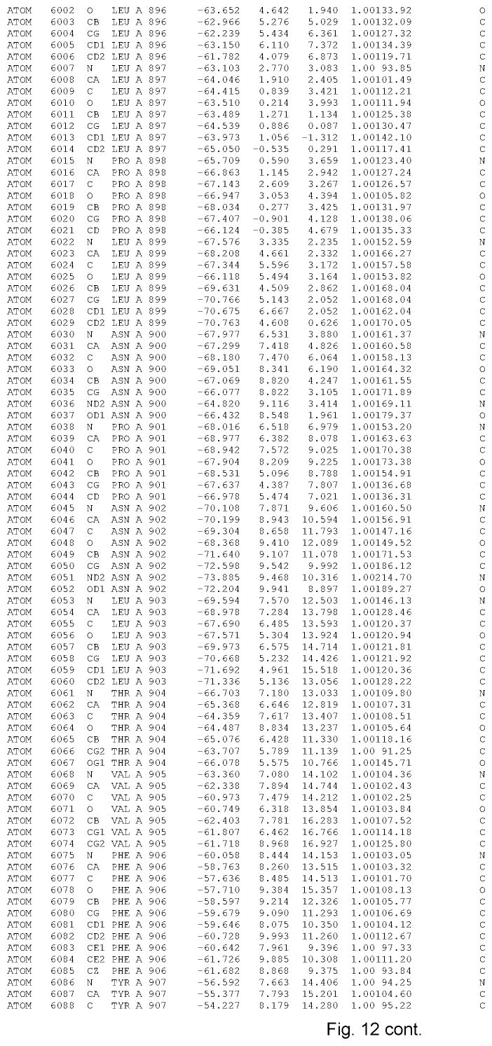

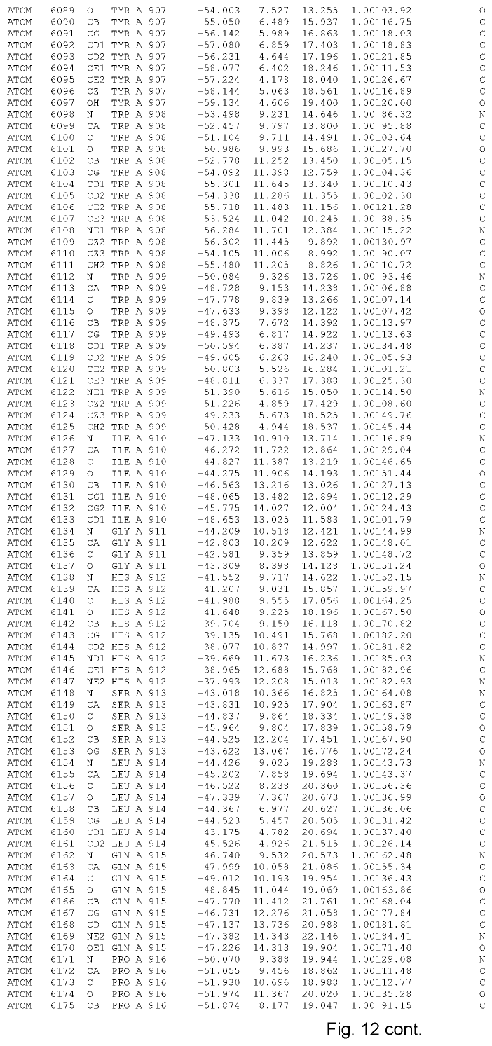

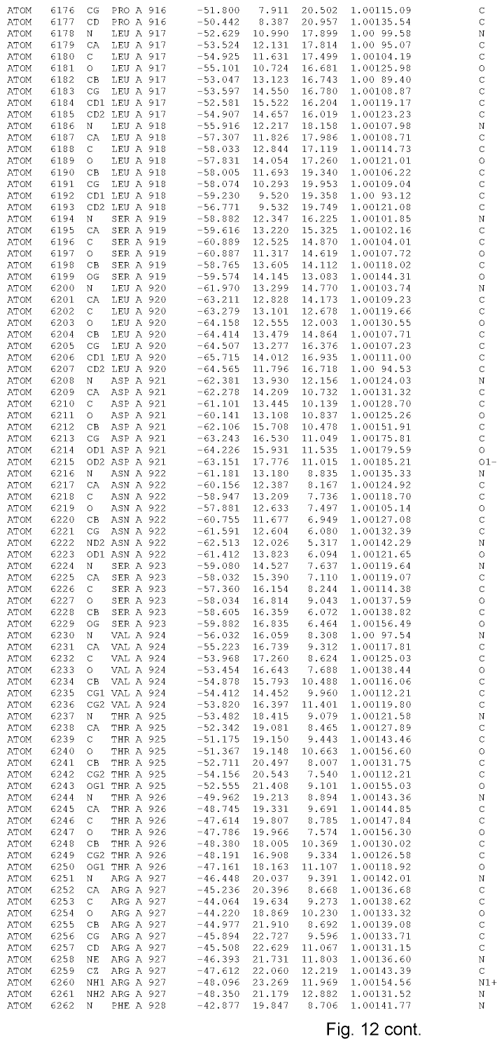

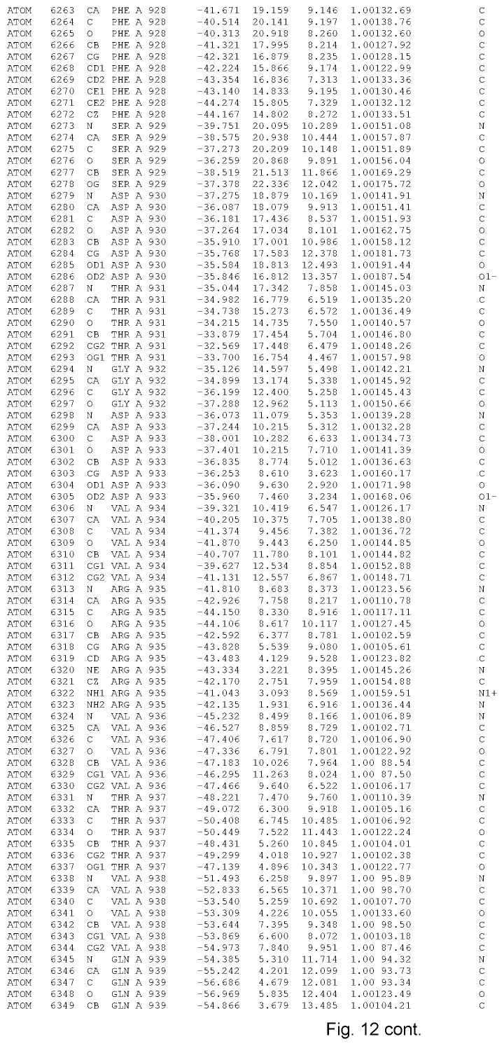

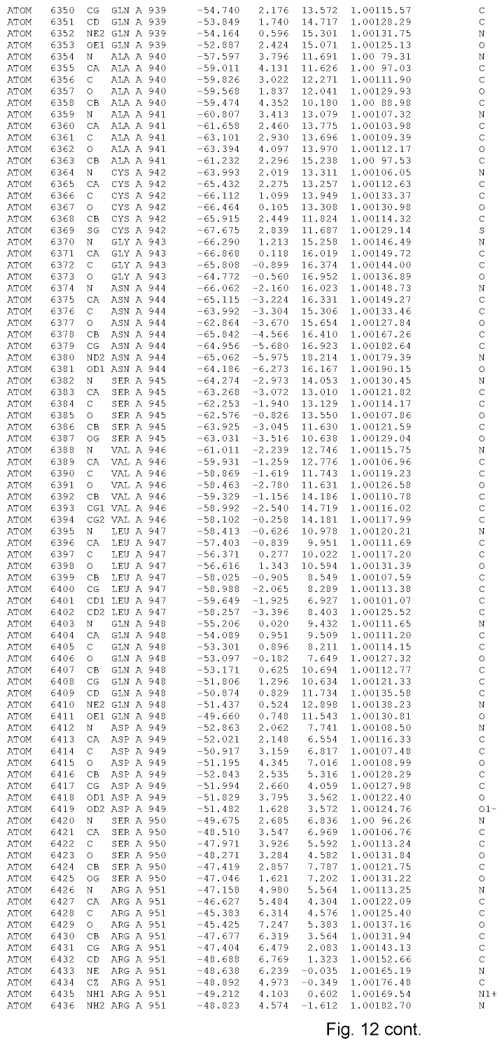

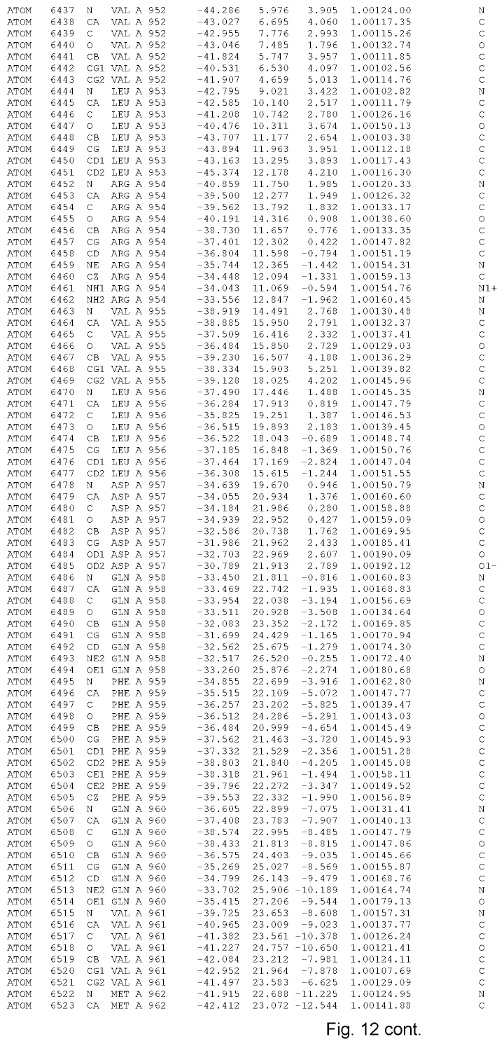

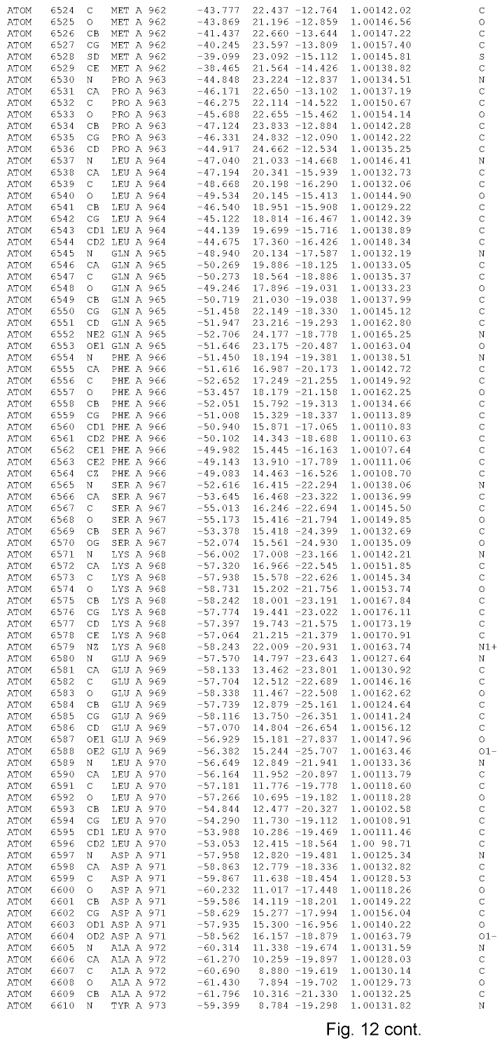

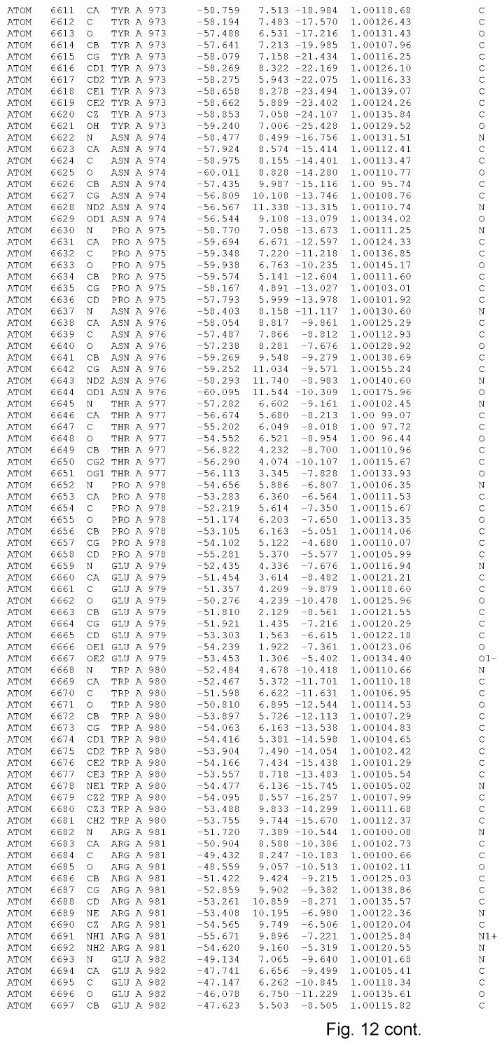

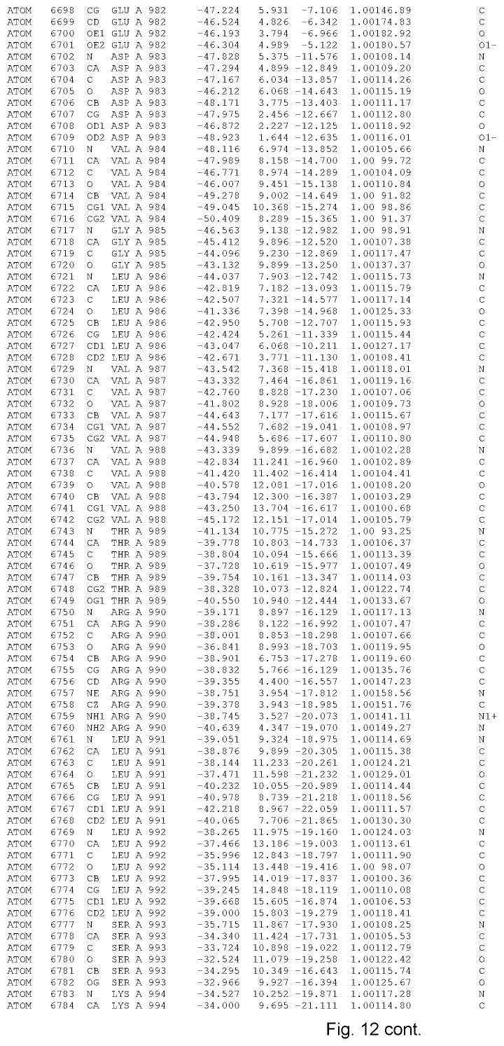

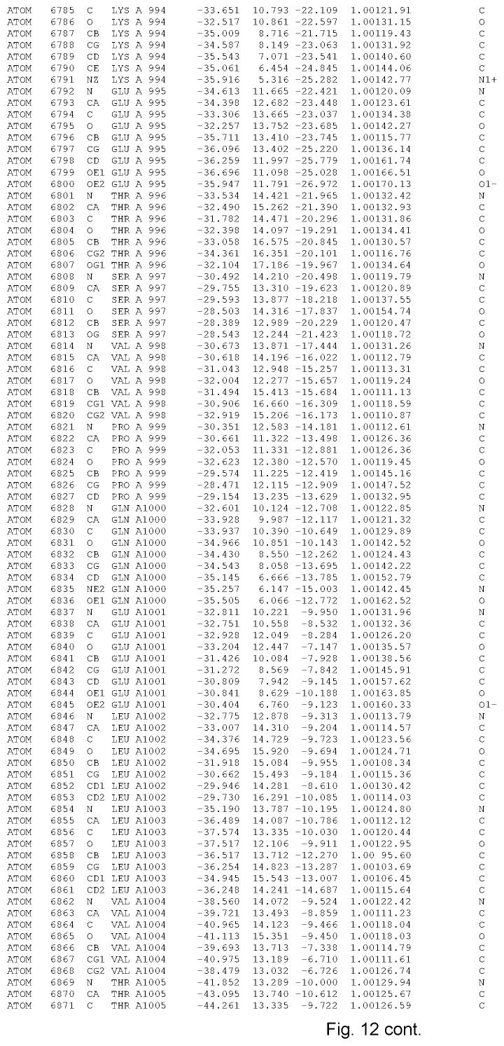

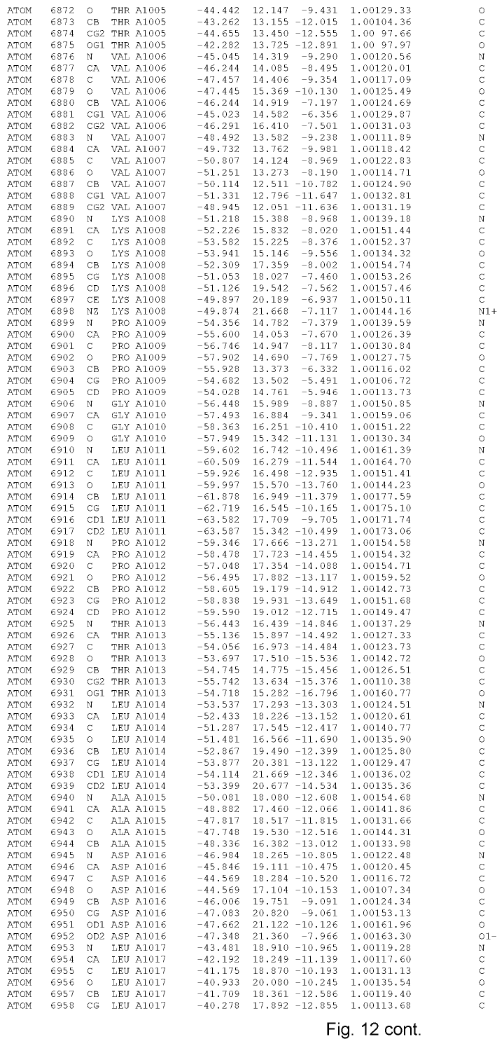

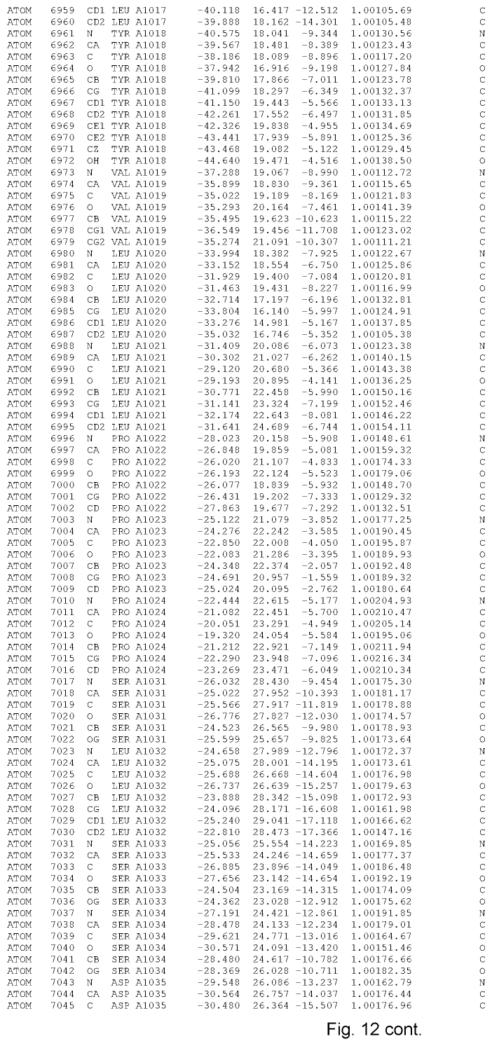

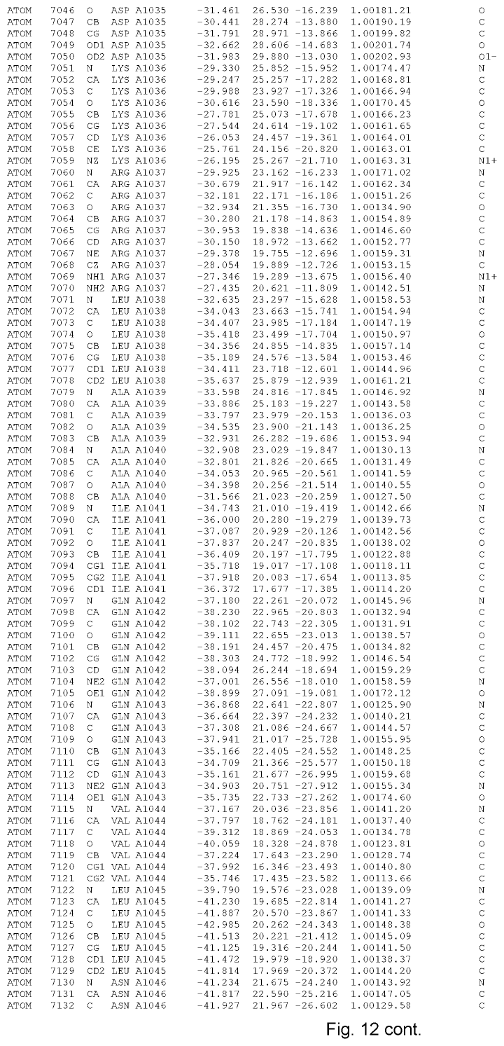

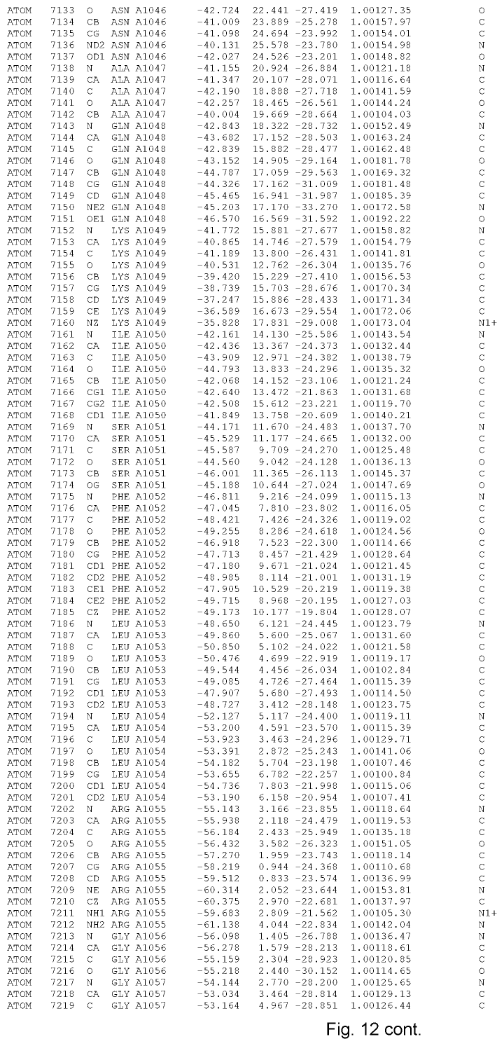

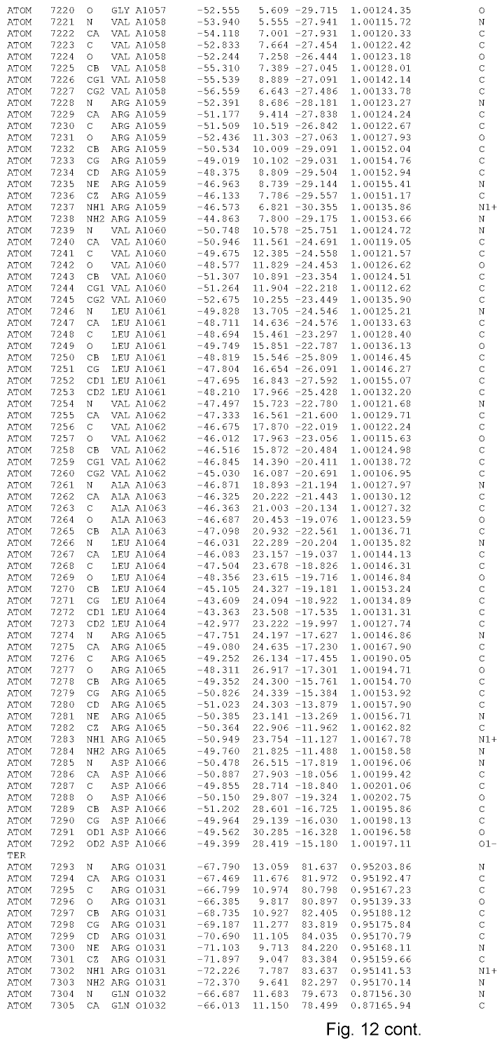

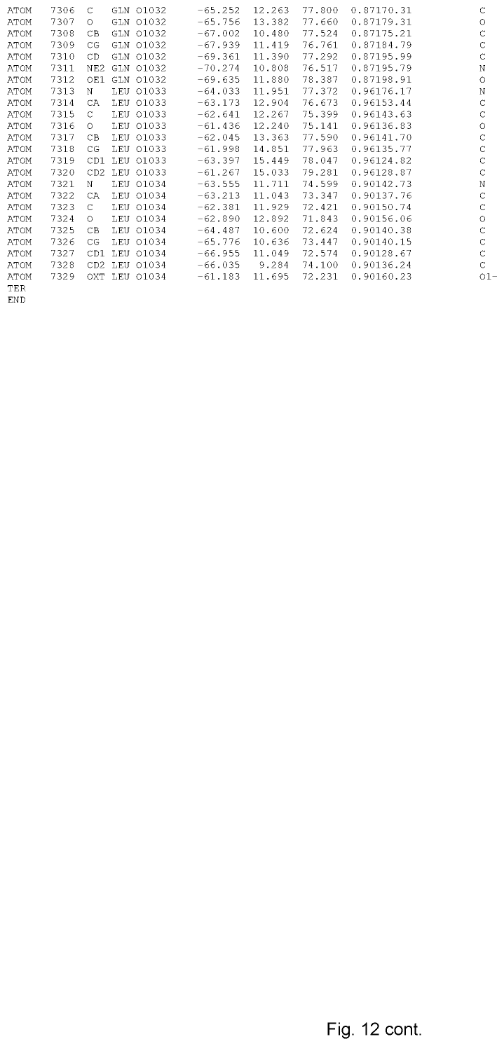

In one aspect, the present disclosure provides a computer-readable data storage medium comprising a data storage material encoded with at least a portion of the structure coordinates set forth in FIG. 12.

Methods for Identifying Ligands

The present disclosure provides screening methods for identification of compounds capable of inhibiting the SorCS2:progranulin binding. In one embodiment, a library of small organic molecules is screened.

In one aspect, the present disclosure relates to use of atomic coordinates as presented in FIG. 12, or atomic coordinates selected from a three-dimensional structure that deviates from the three-dimensional structure as presented in FIG. 12 by a root mean square deviation over protein backbone atoms of not more than 5 Å, in a method for identifying a ligand capable of binding to a β-propeller domain of SEQ ID NO: 1; and/or a fragment or variant thereof.

In one aspect, the present disclosure provides a method of identifying a ligand capable of binding to a β-propeller domain of SEQ ID NO: 1, and/or a fragment or variant thereof, said method comprising the steps of:
a. Generating the spatial structure β-propeller domain of SEQ ID NO: 1 on a computer screen using atomic coordinates as presented in FIG. 12 or atomic coordinates selected from a three-dimensional structure that deviates from the three-dimensional structure that deviates from the three-dimensional structure presented in FIG. 12 by a root mean square deviation over protein backbone atoms of no more than 5 Å;
b. Generating potential ligands with their spatial structure on the computer screen; and
c. Selecting ligands that can bind to at least 1 amino acid residue of the set of binding interaction sites without steric interference.

In one aspect, the present disclosure provides a computer-based method for identifying a ligand capable of binding to a β-propeller domain of SEQ ID NO: 1, and/or a fragment or variant thereof, said method comprising the steps of:
a. Providing a digital embodiment of a three-dimensional structure from the atomic coordinates as presented in FIG. 12, comprising a propeller domain of SEQ ID NO: 1;
b. Generating a digital embodiment of a potential ligand in a computer; and
c. Selecting a ligand that can bind to at least one amino acid residue of the propeller domain of SEQ ID NO: 1.

In one aspect, the present disclosure provides a computer-assisted method for identifying a ligand capable of binding to a β-propeller domain of SEQ ID NO: 1 (SorCS2), and/or a fragment or variant thereof, using a programmed computer comprising a processor, a data storage system, a data input device, and a data output device, said method comprising the steps of:
a. inputting into the programmed computer through said input device data comprising:
   atomic coordinates of a subset of the atoms of said SorCS2, thereby generating a criteria data set;
   wherein said atomic coordinates are selected form at least part of the atomic coordinates data contained in FIG. 12 or atomic coordinates selected from a three-dimensional structure that deviates from the three-dimensional structure presented from the atomic coordinates as presented in FIG. 12, by a root mean square deviation over protein backbone atoms of not more than 5 Å;
b. comparing, using said processor, the criteria data set to a computer data base of low-molecular weight organic chemical structures and peptide fragments stored in the data storage system; and
c. selecting from said data base, using computer methods, a chemical structure having a portion that is structurally complementary to the criteria data set.

In one aspect, the present disclosure provides a method of identifying a potential ligand of a β-propeller domain of SEQ ID NO: 1, and/or a fragment or variant thereof, said method comprising the steps of:
a. introducing into a computer, information derived from atomic coordinates defining a conformation of the β-propeller domain of SEQ ID NO: 1 (SorCS2), and/or a fragment or variant thereof, based on three-dimensional structure determination, whereby a computer program utilizes or displays on the computer screen the structure of said conformation;
   wherein said atomic coordinates are selected from the three-dimensional structure as presented from the atomic coordinates as presented in FIG. 12 or atomic coordinates selected from a three-dimensional structure that deviates from the three-dimensional structure presented from the atomic coordinates as presented in FIG. 12 by a root mean square deviation over protein backbone atoms of not more than 5 Å;
b. generating a three-dimensional representation of the β-propeller domain of SEQ ID NO: 1 by said computer program on a computer screen;
c. superimposing a model of a potential ligand on the representation of said β-propeller domain;
d. assessing the possibility of binding and the absence of steric interference of the potential ligand with the β-propeller domain;
e. incorporating said potential ligand compound in a binding assay of said receptor SorCS2; and
f. determining whether said potential ligand inhibit binding of a competing ligand.

In one embodiment, said competing ligand is progranulin or a fragment thereof. In one embodiment, said progranulin fragment comprises the four most C-terminal amino acid residues of progranulin.

In one aspect, the present disclosure provides a method for identifying a ligand of SorCS2, or a fragment thereof, said method comprising the steps of:
a. Selecting a potential ligand using atomic coordinates in conjunction with computer modelling, wherein said atomic coordinates are the atomic coordinated presented in FIG. 12 or atomic coordinates selected from a three-dimensional structure that deviates from the three-dimensional structure presented from the atomic coordinates as presented in FIG. 12 by a root mean square deviation over protein backbone atoms of not more than 5 Å, by docking potential ligands into a set of binding interaction sites in the β-propeller domain of SEQ ID NO: 1, or a fragment thereof, said binding interaction generated by computer modelling and selecting a potential ligand capable of binding to at least one amino acid residue in said set of binding interaction sites in SorCS2;
b. Providing said potential ligand and said receptor SorCs2;
c. Contacting the potential ligand with said receptor SorCS2; and
d. Detecting binding of said receptor SorCS2 by the potential ligand.

In one embodiment, said ligand of SorCS2, or a fragment thereof, is capable of binding to a β-propeller domain of SEQ ID NO: 1, and/or a fragment or variant thereof. In one embodiment, said ligand binds to at least 1 amino acid residue of the propeller domain of SEQ ID NO: 1, such as at least 2 amino acid residues, such as at least 3 amino acid residues, such as at least 4 amino acid residues, such as at least 5 amino acid residues of the propeller domain of SEQ ID NO: 1. In one embodiment, said ligand is capable of binding to at least one amino acid residue selected from the group consisting of amino acid residues 528, 459, 468, 475 to 479, 663, and 704 of SEQ ID NO: 1. In one embodiment, said ligand is capable of binding to at least one amino acid residue, such as 1, such as 2, such as 3, such as 4, such as 5, such as 6, such as 7, such as 9, such as 10 amino acid residues selected from the group consisting of amino acid residues 528, 459, 468, 475 to 479, 663, and 704 of SEQ ID NO: 1.

In one embodiment, the potential ligand is selected from the group consisting of non-hydrolyzable peptides and peptide analogues, organic compounds and inorganic compounds.

In one embodiment, the atomic coordinates are determined to a resolution of 4.5 Å or less, such as 4 Å or less, such as 3 Å or less, such as 2 Å or less, such as 1.5 Å or less.

Agents

In one aspect, the present disclosure relates to an agent comprising or consisting of the sequence RQLL (SEQ ID NO: 2), wherein said agent comprises no more than 20 amino acid residues. In one embodiment, said agent is a peptide. The agent may be glycosylated and/or lipidated and/or comprise prosthetic groups. In one embodiment, said the agent is a peptide conjugated to a moiety.

In one embodiment, the agent comprises no more than 19 amino acid residues, such as no more than 18 amino acid residues, such as no more than 17 amino acid residues, such as no more than 16 amino acid residues, such as no more than 15 amino acid residues, such as no more than 14 amino acid residues, such as no more than 13 amino acid residues, such as no more than 12 amino acid residues, such as no more than 11 amino acid residues, such as no more than 10 amino acid residues, such as no more than 9 amino acid residues, such as no more than 8 amino acid residues, such as no more than 7 amino acid residues, such as no more than 6 amino acid residues, such as no more than 5 amino acid residues.

In one embodiment, the agent comprises at least 5 amino acid residues, such as at least 6 amino acid residues, such as at least 7 amino acid residues, such as at least 8 amino acid residues, such as at least 9 amino acid residues, such as at least 10 amino acid residues, such as at least 11 amino acid residues, such as at least 12 amino acid residues, such as at least 13 amino acid residues, such as at least 14 amino acid residues, such as at least 15 amino acid residues, such as at least 16 amino acid residues, such as at least 17 amino acid residues, such as at least 18 amino acid residues, such as at least 19 amino acid residues.

In one embodiment, the number of amino acid residues of the agent is in the range of 4 to 20 amino acid residues, such as 4 to 15 amino acid residues, such as 4 to 10 amino acid residues, such as 4 to 8 amino acid resides.

In one embodiment, the agent is a peptide of the sequence RQLL (SEQ ID NO: 2).

In one embodiment, said agent is a ligand of SorCS2 (SEQ ID NO: 1), such as a ligand identified as described above. In one embodiment, said agent is capable of binding to a β-propeller domain of SEQ ID NO: 1 and/or a fragment or variant thereof. In one embodiment, said agent is capable of binding to at least 1 amino acid residue of the propeller domain of SEQ ID NO: 1, such as at least 2 amino acid residues, such as at least 3 amino acid residues, such as at least 4 amino acid residues, such as at least 5 amino acid residues of the propeller domain of SEQ ID NO: 1. In one embodiment, said agent is capable of binding to at least one amino acid residue selected from the group consisting of amino acid residues 528, 459, 468, 475 to 479, 663, and 704 of SEQ ID NO: 1. In one embodiment, said agent is capable of binding to at least one amino acid residue, such as 1, such as 2, such as 3, such as 4, such as 5, such as 6, such as 7, such as 9, such as 10 amino acid residues selected from the group consisting of amino acid residues 528, 459, 468, 475 to 479, 663, and 704 of SEQ ID NO: 1. In one embodiment, the agent is capable of inhibiting the direct interaction between hSorCS2 and hPGRN. Hence, in one embodiment, the agent is capable of reducing the extracellular levels of progranulin. In one embodiment, the agent is capable of reducing the intracellular levels of progranulin and/or granulins.

Medical Use

It is well established that reduced plasma levels of PGRN lead to frontotemporal dementia (FTD). Thus, methods for elevating the plasma levels of PGRN may provide treatment, alleviation and/or prevention of FTD. By means of affinity chromatography, mass spectrometry, and surface plasmon resonance (SPR) and microscale thermophoresis (MST) analysis, the present inventors have demonstrated that PGRN interacts with SorCS2, and that this interaction appears to play a significant and important role, i.e. that SorCS2 reduces absolute levels of PGRN as well as processed granulins. Hence, inhibition of the SorCS2:PGRN interaction may elevate the levels of plasma PGRN and thus be useful in the treatment of FTD.

As used herein, the term “frontotemporal dementia” refers to a group of disorders caused by progressive nerve cell loss in the brain's frontal lobes or its temporal lobes, and the term is exchangeable with “frontotemporal lobar degeneration”, “FTD” and “FTLD”.

In one aspect, the present disclosure relates to the agent for use as a medicament.

In one aspect, the present disclosure relates to the agent for use in a method for treating frontotemporal dementia (FTD), wherein said method comprises administering said agent to a subject in need thereof. In one embodiment, the subject is a mammal, such as a human.

In one aspect, the present disclosure relates to a method for increasing the extracellular levels of progranulin, said method comprising administering an agent as defined herein.

### EXAMPLES

### Example 1: Progranulin Co-Purifies with hSorCS2

hSorCS2 produced in CHO-K1 cells and purified as described in example 2 and purified on Talon beads (Clontech) from cell culture medium was visualized by SDS-PAGE and Coomassie staining. A putative ligand for SorCS2 copurifying with hSorCS2 was excised from the gel. Following in-gel trypsin digest and peptide analysis by MALDI 13 peptides from the putative ligand were identified as progranulin expressed by the CHO-K1 cells; 4 peptides were further verified by MS/MS sequencing. The analysis was carried out by Alphalyse (Odense, Demark).

The results show that human SorCS2 interacts with hamster progranulin.

### Example 2: Expression of hSorCS2

The cDNA encoding the extracellular domain of human SorCS2 and a C-terminal ₆His-tag was inserted into the pCpGfree-vitroNmcs vector and transformed into the E. coli strain GT115 encoding the pir gene (invivogen). DNA sequence integrity was confirmed by sanger sequencing (Eurofins). The episomal plasmid was transfected into mammalian CHO-K1 cells and stable clones were selected using G418. Cells expressing SorCS2 were subsequently adapted to soluble growth in Hybridoma-SFM medium (Gibco) and expanded in 3-layer flasks (Thermo Fisher Scientific).

### Example 3: hSorCS2 Purification and Deglycosylation

Recombinant expressed protein was purified from the cell culture medium using Talon beads (Clontech) according to the manufacturer's instructions. HSorCS2 was deglycosylated over night at room temperature by home-made PNGaseF (1:10 m/m ratio). Deglycosylation was monitored by SDS-PAGE and SorCS2 was further purified by Ni2+ affinity chromatography and finally gel-filtrated on a Superdex200 increase 10/300 column (GEhealtcare) in 25 mM Tris pH 7.4 and 150 mM NaCl.

### Example 4: Crystallization and Cryoprotection

Hanging and sitting drop vapour-diffusion experiments were set up in 24 well plates at 292 K. In these screens, 1 ul reservoir solution was added to 1 ul protein solution (25 mM Tris pH 7.4 and 150 mM NaCl) and allowed to equilibrate against 490 ul reservoir solution. Crystals were formed in a mother-liquor cocktail of 10-15% PEG6000 and 0-0.3 M MgCl₂ and 0.1 M Sodium Citrate pH 5.5. Initial crystals formed interconnected needles and were optimized by a combination of lowering the protein concentration to 1-2 gl⁻¹ and streak seeding into clear drops. Following this procedure, single protein crystals formed after 4-10 days. Crystals were cryo-protected in reservoir solution supplemented with either glycerol, sucrose or PEG400 to 15-25% and flash cooled in liquid nitrogen. Crystals of hSorCS2 in complex with a C-terminal peptide fragment of PGRN were obtained by soaking native hSorCS2 crystals in cryo-protectant buffered to pH 6.5 and supplemented with peptide to 2 mM.

### Example 5: X-Ray Diffraction Data Collection and Model Refinement

X-ray diffraction images were collected at 100 K on beam line ID23-1 at ESRF or at BioMax at MAXIV. Images were processed using XDS. The processing included integration using XDS, data scaling using XSCALE and the final conversion to MTZ format using XDSCONV. Phase determination was performed by molecular replacement using the program Phenix.Phaser by placing two copies of the SorLa Vps10p domain (PDBid: 3WSZ). The initial electron density map phased by the MR solution was used for density modification and automatic model building using the program Resolve in Phenix.Autobuild. The electron density after Autobuild showed additional density outside the Vps10p domains. Further rounds of automatic model building using Resolve and Buccaneer combined with manual editing in Coot further improved the electron density and a near complete model of the SorCS2 dimer in the asymmetric unit was obtained for hSorCS2apo containing a total of 21 sugar residues.

At this point, all glycans present in the model were removed and the resultant structure was used for MR of hSorCS2:RQLL crystals. The obtained solution contained two dimers in the asymmetric unit and was subjected to five rounds of refinement in Phenix.refine which included simulated annealing to remove model bias. Additional electron density accounting for the removed glycans were clearly visible in the Fo-Fc electron density map confirming the integrity of this solution. SorCS2:RQLL was finally refined by iterative rounds of real-space refinement and model fitting in Coot and reciprocal-space refinement using Phenix. Refine. Additional electron density accounting for the PGRN derived peptide was clearly visible on all monomers of hSorCS2 within this crystal form. The binding site is novel within the Vps10p receptor family as it is situated at a different binding groove then previously described for Sortilin, SorLA and mSorCS2. The resultant diffraction and refinement statistics are presented in Table 1.

TABLE 1
Data collection and refinement statistics for hSorCS2.

| Data Collection | hSorCS2-apo | | hSorCS2:RQLL | |
|---|---|---|---|---|
| Beamline | ESRF id 23-1 | | MAX IV BioMax | |
| Angle increment [°] | 0.1 | | 0.1 | |
| Wavelength [Å] | 1.00 | | 0.979 | |
| Resolution [Å] | 50-3.2 | (3.4-3.2) | 46-3.3 | (3.42-3.3) |
| Space group | P12₁1 | | P2₁2₁2₁ | |
| a, b, c [Å] | 82.6, 134.6, 146.3 | | 137.3, 147.6, 310 | |
| α, β, γ [°] | 90, 100.7, 90 | | 90.0, 90.0, 90.0 | |
| Measured reflections | 124538 | (18397) | 889007 | (66783) |
| Unique reflections | 51678 | (8032) | 97040 | (7087) |
| Multiplicity | 2.4 | (2.3) | 9.2 | (9.4) |
| Completeness [%] | 99.5 | (99.1) | 99.9 | (100) |
| I/<σ(I) | 5.6 | (1.0) | 7.4 | (1.24) |
| CC1/2 (%) | 97.9 | (57.9) | 99.7 | (67.5) |
| Rmeas [%] | 21.1 | (103.0) | 23.9 | (165.6) |

| | | |
|---|---|---|
| Wilson B [Å²] | 72.16 | 89.3 |

Refinement

| | | | | |
|---|---|---|---|---|
| Rwork | 0.237 | (0.426) | 0.243 | (0.368) |
| Rfree | 0.277 | (0.439) | 0.2714 | (0.380) |

Average B-factors

| | | |
|---|---|---|
| Protein | 80.8 | 109.9 |
| Glycosylations | 114.5 | 132.9 |

Number of non-hydrogen atoms

| | | |
|---|---|---|
| Protein | 14562 | 29238 |
| Glycosylations | 262 | 569 |
| RMS (bonds) | 0.03 | 0.008 |
| RMS (angles) | 0.6 | 1.09 |
| Ramachandran favored [%] | 94.8 | 89.8 |
| Ramachandran allowed [%] | 4.78 | 8.14 |
| Ramachandran outliers [%] | 0.44 | 2.1 |
| Rotamer outliers [%] | 5.0 | 0.09 |

Statistics for the highest-resolution shell are shown in parentheses.

### Example 6: Physical Interaction Between SorCS2 and Progranulin

6.1 Surface Plasmon Resonance

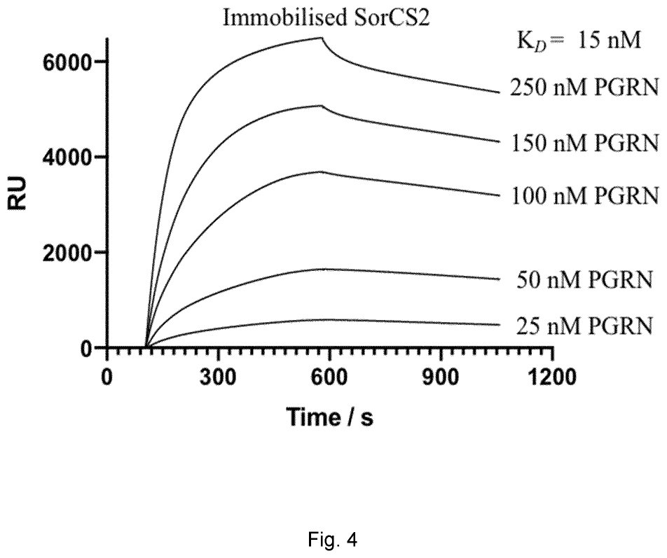

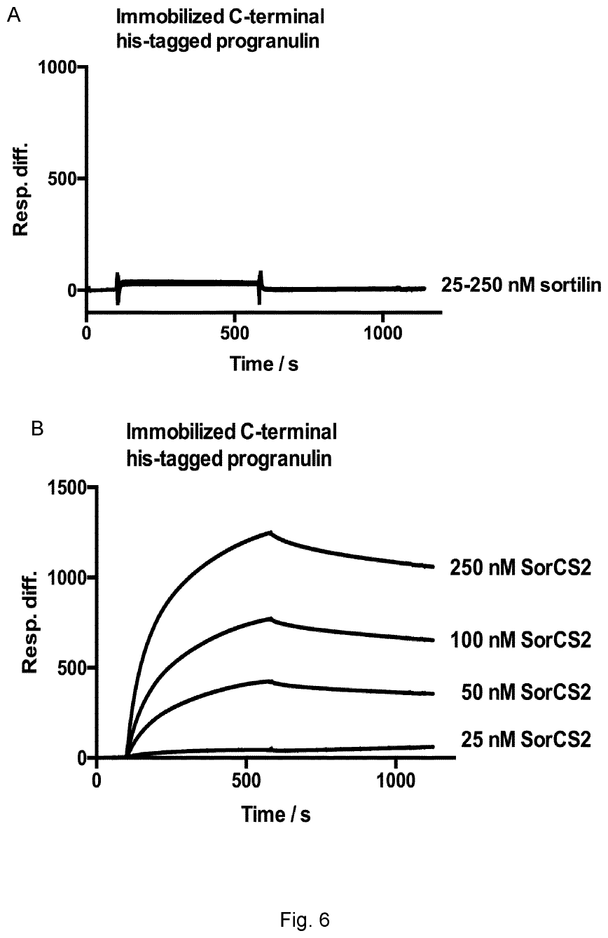

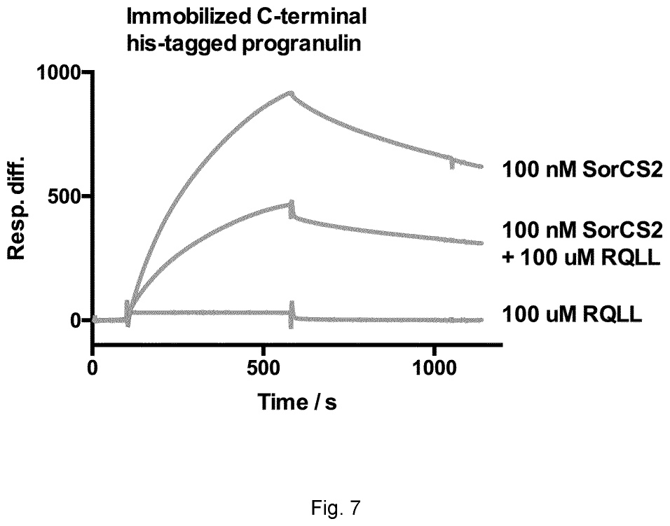

Purified hPGRN-6his or hSorCS2-6his was immobilized on a CM5 sensor chip using standard amine-coupling. All sensograms were collected in PBS+0.05% Tween20 following injection of purified hPGRN-6his over immobilized hSorCS2-6his or hSortilin-6his and hSorCS2-6his with/without PGRN derived peptide over immobilized hPGRN-6his. Following this experimental setup we estimated the dissociation constant of the hSorCS2:hPGRN interaction to be 15 nM (FIG. 4). Using a similar experimental setup, we found that hSorCS2 interacted with immobilized hPGRN-6his and the dissociation constant was estimated to 7.4 nM (FIG. 6). Inhibition experiments was performed using 100 nM hSorCS2 preincubated with 100 uM PGRN-derived tetra peptide and injected over immobilized hPGRN-6his (FIG. 7).

6.2 Microscale Thermophoresis

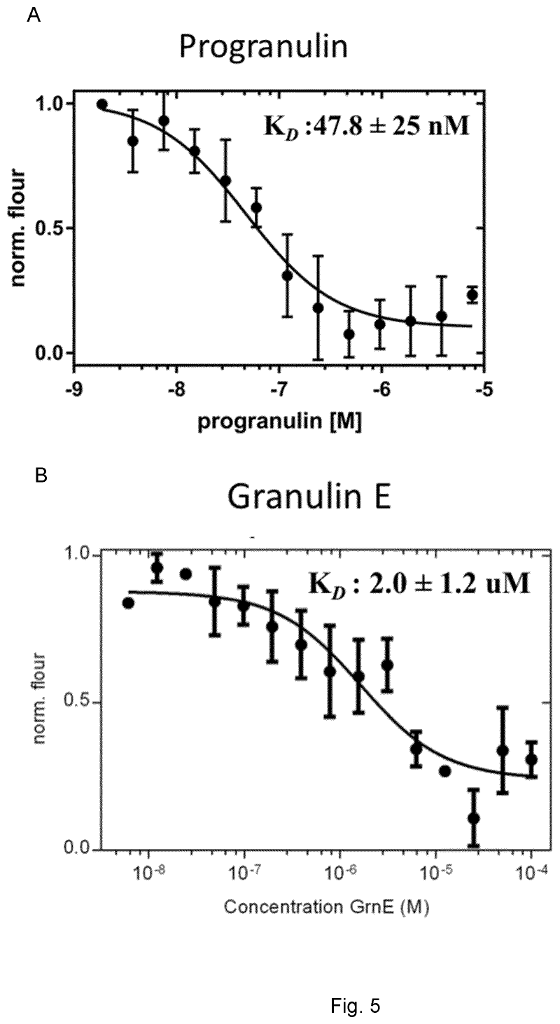

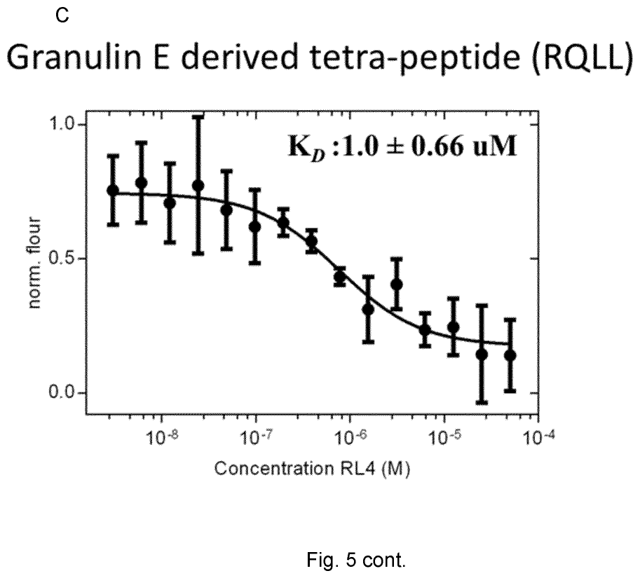

Equilibrium-binding affinities between hSorCS2 and hPGRN-6his, GrnE and RQLL peptide were assessed by MST. Purified hSorCS2-6his was labelled using the MO-003 monolith blue-NHS labelling kit according to manufactures recommendation. hSorCS2 was applied at a final concentration of 25 nM while the unlabelled binding partner was titrated in 1:1 dilutions (PBS+0.05% Tween20). MST measurements were performed in standard treated capilaiers on a monolith NT.115 instrument. For each binding partner the sigmoid-dose response curve was fitted with GraphPad prism 6 to yield an average dissociation constant. All experiments were performed in triplicates and the estimated K_{D} values were 47.8 nM (hPGRN-6his), 2 uM (GrnE) and 1 uM (RQLL) (FIG. 5).

6.3 Conclusions

The presented binding experiments suggest a direct physical interaction between hSorCS2 extracellular domain and hPGRN. The main binding site of progranulin is located in granulin E and includes the four most C-terminal amino acids with the amino acid sequence “ROLL”. Sortilin, which relies on progranulin C-terminal amino acids for binding, requires a free C-terminal carboxyl group for the interaction. In contrast, SorCS2 does not require a free C-terminal carboxylate group to be present on the terminal Leucine of progranulin. We further found that the C-terminal PGRN derived peptide can partially inhibit the direct interaction between hSorCS2 and hPGRN.

### Example 7: SorCS2 Affects Subcellular Distribution of Progranulin in Mice CNS

By sucrose gradient ultracentrifugation experiments of whole brain preparations of WT and SorCS2 knock out mice, the effect of SorCS2 on subcellular distribution of progranulin has been assessed. Brains were homogenized in ice-cold Solution A (0.25 mM sucrose, 1 mM EDTA, 20 mM HEPES-KOH, pH 7.4 plus Complete (Roche)) and centrifuged for 10 min at 1000×g followed by another centrifugation at 3000×g for 10 min. The resulting supernatant was then ultracentrifuged for 10 min at 13,000 rpm (Ti70.1 rotor) and again at 50,000 rpm for 45 min. Pellet was dissolved in Solution A and overlaid a continuous 0.8-1.6 M sucrose gradient (in 10 mM HEPES-KOH pH 7.2) prepared by using a Biocomp gradient master. Samples were ultracentrifuged at 25000 rpm for 18 hours using a SW40 rotor and fractions collected using a Biocomp piston gradient fractionator. The resulting fractions were analyzed by western blotting.

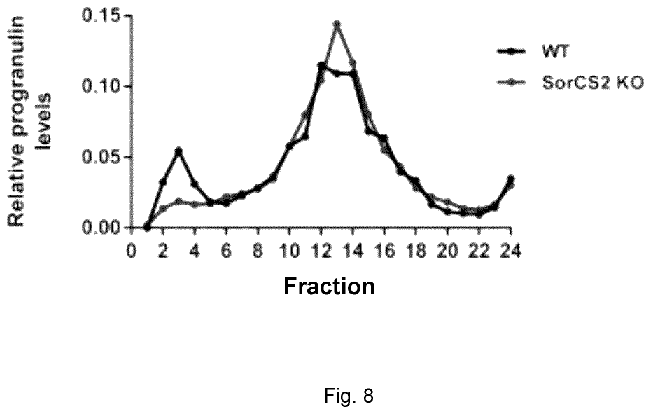

Lack of SorCS2 redistribute progranulin from light-density fractions (fraction 2, 3, and 4) to higher-density fractions (fraction 12, 13, and 14) (FIG. 8).

The results show that SorCS2 affects progranulin subcellular distribution in mouse CNS.

### Example 8: SorCS2 Decreases Extracellular and Intracellular Levels of Progranulin and Granulins

The effect of SorCS2 on progranulin and granulin levels in human embryonic kidney cells (HEK293 cells) was addressed by western blotting of cell lysates and media. Cells were transiently transfected with fuGENE 6 (Promega) using 3.2 uL/mL fuGENE and 0.8 ug/mL cDNA. 24 hours post transfection, media were collected and cells lyzed in THE lysis buffer (20 mM Tris pH 8, 10 mM EDTA, 1% NP40, complete protease inhibitor cocktail (Roche)) on ice for 10 min.

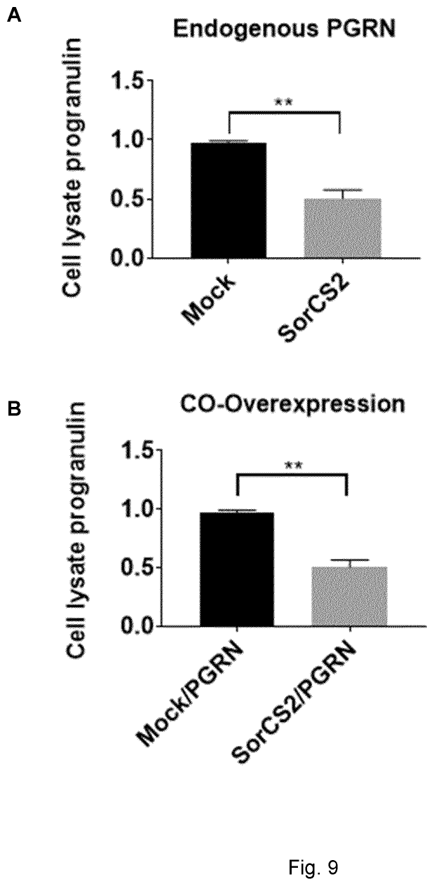

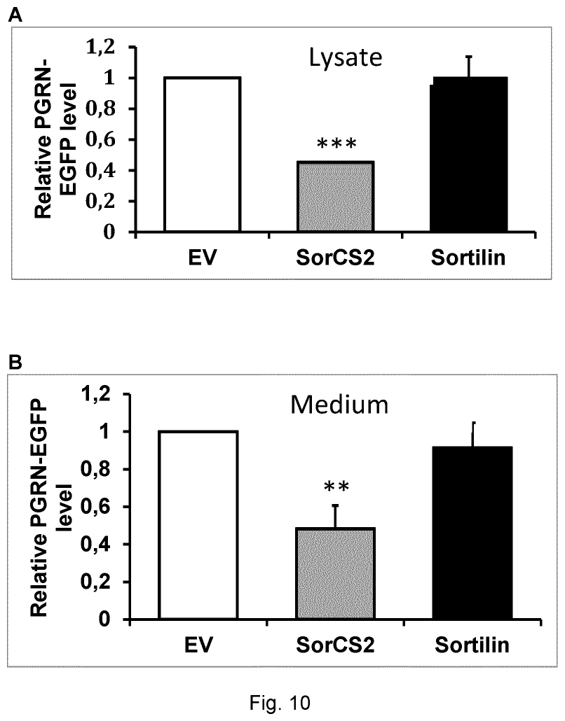

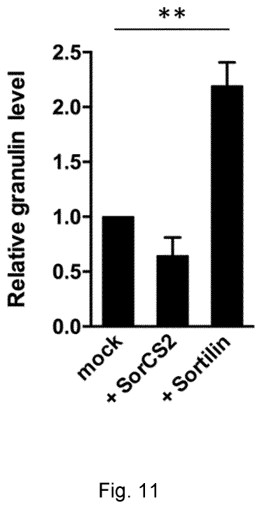

SorCS2 reduced extracellular and intracellular levels of overexpressed progranulin (FIG. 9) and accordingly generation of granulins in cells (FIG. 11). Likewise, endogenous level of extracellular and intracellular progranulin was reduced by transfection with SorCS2. Similarly, co-transfection with SorCS2 reduced extracellular and intracellular levels of C-terminally EGFP-tagged progranulin in cells (FIG. 10). There was only a minor effect on C-terminally EGFP-tagged progranulin by transfection with sortilin.

Data show that SorCS2 reduces extracellular levels of progranulin and intracellular levels of progranulin and granulins in HEK293 cells both overexpressed and endogenous. For SorCS2, the capacity to reduce progranulin levels in cells does not require a free C-terminal carboxyl group of progranulin.

Sequences

hSorCS2
SEQ ID NO: 1
MAHRGPSRASKGPGPTARAPSPGAPPPPRSPRSRPLLLLLLLLGACGAAGR
SPEPGRLGPHAQLTRVPRSPPAGRAEPGGGEDRQARGTEPGAPGPSPGPAP
GPGEDGAPAAGYRRWERAAPLAGVASRAQVSLISTSFVLKGDATHNQAMVH
WTGENSSVILILTKYYHADMGKVLESSLWRSSDFGTSYTKLTLQPGVTTVI
DNFYICPTNKRKVILVSSSLSDRDQSLFLSADEGATFQKQPIPFFVETLIF
HPKEEDKVLAYTKESKLYVSSDLGKKWTLLQERVTKDHVFWSVSGVDADPD
LVHVEAQDLGGDFRYVTCAIHNCSEKMLTAPFAGPIDHGSLTVQDDYIFFK
ATSANQTKYYVSYRRNEFVLMKLPKYALPKDLQIISTDESQVFVAVQEWYQ
MDTYNLYQSDPRGVRYALVLQDVRSSRQAEESVLIDILEVRGVKGVFLANQ
KIDGKVMTLITYNKGRDWDYLRPPSMDMNGKPTNCKPPDCHLHLHLRWADN
PYVSGTVHTKDTAPGLIMGAGNLGSQLVEYKEEMYITSDCGHTWRQVFEEE
HHILYLDHGGVIVAIKDTSIPLKILKFSVDEGLTWSTHNFTSTSVFVDGLL
SEPGDETLVMTVFGHISFRSDWELVKVDFRPSFSRQCGEEDYSSWELSNLQ
GDRCIMGQQRSFRKRKSTSWCIKGRSFTSALTSRVCECRDSDFLCDYGFER
SSSSESSTNKCSANFWFNPLSPPDDCALGQTYTSSLGYRKVVSNVCEGGVD
MQQSQVQLQCPLTPPRGLQVSIQGEAVAVRPGEDVLFVVRQEQGDVLTTKY
QVDLGDGFKAMYVNLTLTGEPIRHRYESPGIYRVSVRAENTAGHDEAVLFV
QVNSPLQALYLEVVPVIGLNQEVNLTAVLLPLNPNLTVFYWWIGHSLQPLL
SLDNSVTTRFSDTGDVRVTVQAACGNSVLQDSRVLRVLDQFQVMPLQFSKE
LDAYNPNTPEWREDVGLVVTRLLSKETSVPQELLVTVVKPGLPTLADLYVL
LPPPRPTRKRSLSSDKRLAAIQQVLNAQKISFLLRGGVRVLVALRDTGTGA
EQLGG
SEQ ID NO: 2
ROLL

### REFERENCES

1 Kao, A. W. et al. (2017) Nature Reviews Neuroscience 18: 325-33
2 Baker, M. et al. (2006) Nature 442: 916-9
3 Cruts, M. et al. (2006) Nature 442: 920-4
4 Neumann, M. (2006) Science 314: 130-3
5 Smith, K. R. et al. (2012) American Journal of Human Genetics 90: 1102-7
6 Almeida, M. R. et al. (2016) Neurobiology of Aging 41: 200.e1e200.e5
7 Ward, M. E. (2017 Science Translational Medicine 9: https://DOI.org/10.1126/scitranslmed.aah5642
8 Götzl, J. K. et al. (2014) Acta Neuropathologica 127: 845-60
9 Hu, F. et al. (2010) Neuron 68: 654-67
10 Holler, C. J. et al. (2017) Intracellular proteolysis of progranulin generates stable, lysosomal granulins that are haploinsufficient in patients with frontotemporal dementia caused by GRN mutations. eNeuro 4: 1-22

1. A crystal comprising
a) a polypeptide comprising the amino acid sequence of SEQ ID NO: 1;
b) a biologically active sequence variant of the polypeptide of a), wherein the variant has at least 90% sequence identity to SEQ ID NO: 1; and/or
c) a biologically active fragment comprising at least 500 contiguous amino acids of any of a) through b);
wherein the biological activity is SorCS2 activity.

2. The crystal according to any one of the preceding claims, wherein the crystal is in complex with at least one ligand.

3. The crystal according to any one of the preceding claims, wherein the at least one ligand is bound to a progranulin binding site comprising at least one of amino acid residues 528, 459, 468, 475 to 479, 663, and 704 of SEQ ID NO: 1.

4. The crystal according to any one of the preceding claims, wherein the sequence variant of the polypeptide has at least 91% sequence identity to SEQ ID NO: 1, such as 92% identity, such as 93% identity, such as 94% identity, such as 95% identity, such as 96% identity, such as 97% identity, such as 98% identity, such as 99% identity to SEQ ID NO: 1.

5. The crystal according to any one of the preceding claims, wherein the amino acid sequence further comprises one or more C-terminal histidine residues.

6. The crystal according to any one of the preceding claims, wherein the crystal is of orthorhombic of monoclinic space group.

7. The crystal according to any one of the preceding claims, wherein the crystal is P12₁1 or P2₁2₁2₁.

8. The crystal according to any one of the preceding claims, wherein the unit cell parameters are a=137.3±3 Å, b=147.6±3 Å, c=310±3 Å, and α=β=γ=90°.

9. The crystal according to any one of the preceding claims, wherein the unit cell parameters are a=137.3 Å, b=147.6 Å, c=310 Å, and α=β=γ=90°.

10. The crystal according to any one of the preceding claims, wherein the unit cell parameters are a=82.6±3 Å, b=134.6±3 Å, c=146.3247.6±3 Å, and α=90°, β=100.7°, γ=90°.

11. The crystal according to any one of the preceding claims, wherein the unit cell parameters are a=82.6 Å, b=134.6 Å, c=146.3247.6 Å, and α=90°, β=100.7°, γ=90°.

12. A method of growing the crystal according to any one of the preceding claims, comprising the steps of:
a. providing a protein composition comprising a polypeptide of SEQ ID NO: 1 or a fragment or variant thereof;
b. providing a composition comprising a precipitant;
c. arranging an equilibrium between the protein composition of a with the precipitant composition of b in a container; and
d. obtaining a crystal comprising the polypeptide of SEQ ID NO: 1 or the fragment or variant thereof.

13. The method according to claim 12, wherein the temperature within the container is between 1 and 30° C., such as between 10 and 25° C., such as between 15 and 20° C., such as between 18 and 20° C., such as 19° C.

14. The method according to any one of claims 12 to 13, wherein the protein composition comprises Tris pH 7.2-7.8, such as Tris pH 7.4.

15. The method according to any one of claims 12 to 14, wherein protein composition comprises in the range of 10 to 100 mM Tris, such as in the range of 10 to 50 mM, such as in the range of 15 to 40 mM, such as in the range of 20 to 30 mM, for example 25 mM Tris.

16. The method according to any one of claims 12 to 15, wherein the protein composition comprises in the range of 10 to 500 mM NaCl, such as in the range of 50 to 250 mM, such as in the range of 100 to 200 mM, such as in the range of 125 to 175 mM, such as 150 mM NaCl.

17. The method according to any one of claims 12 to 16, wherein the precipitant composition comprises at least one component selected from the group consisting of: sodium citrate, magnesium chloride, and polyethylene glycol (PEG).

18. The method according to any one of claims 12 to 17, wherein the precipitant composition comprises sodium citrate, such as in the range of 10 to 500 mM sodium citrate, for example 100 mM sodium citrate.

19. The method according to any one of claims 12 to 18, wherein the precipitant composition comprises sodium citrate pH 5.5.

20. The method according to any one of claims 12 to 19, wherein the precipitant composition comprises magnesium chloride, such as in the range of 10 to 500 mM magnesium chloride, for example 300 mM magnesium chloride.

21. The method according to any one of claims 12 to 20, wherein the precipitant composition comprises PEG with an average molecular weight from 3350 to 20000 Da.

22. The method according to any one of claims 12 to 21, wherein the precipitant composition comprises PEG selected from the group consisting of: PEG 3350, PEG 4000, PEG 6000, and PEG 8000.

23. The method according to any one of claims 12 to 22, wherein the precipitant composition comprises in the range of 5 to 20% w/v PEG 6000, such as in the range of 7 to 18% w/v PEG 6000, such as in the range of 9 to 16% w/v PEG 6000, such as in the range of 10 to 15% w/v PEG 6000.

24. The method according to any one of claims 12 to 23, wherein the ratio of volumes between protein composition and precipitant composition is less than 5 to 1, such as less than 4 to 1, such as less than 3 to 1, such as less than 2 to 1, such as 1:1.

25. The method according to any one of claims 12 to 24, wherein the initial concentration of the polypeptide in the protein composition is between 0.1 to 10 mg/mL, such as between 0.1 and 9 mg/mL, such as between 0.1 and 8 mg/mL, such as between 0.1 and 7 mg/mL, such as between 0.1 and 6 mg/mL, such as between 0.5 and 5 mg/mL, such as between 0.5 and 2.5 mg/mL, such as between 1 to 2 mg/mL.

26. The method according to any one of claims 12 to 25, further comprising the steps of:
a. isolating a crystalline precipitate, and
b. growing the crystalline precipitate by vapor diffusion from hanging drops or sitting drops.

27. Use of a crystal according to any one of the preceding claims for determination of the three dimensional structure of SorCS2 or a fragment or variant thereof.

28. The use according to claim 27, wherein the crystal further comprises a ligand bound to SorCS2 for determination of the three dimensional structure of SorCS2 or a fragment or variant thereof in complex with said ligand.

29. A computer-readable data storage medium comprising a data storage material encoded with at least a portion of the structure coordinates set forth in FIG. 12.

30. Use of atomic coordinates as presented in FIG. 12 or atomic coordinates selected from a three-dimensional structure that deviates from the three-dimensional structure as presented in FIG. 12 by a root mean square deviation over protein backbone atoms of not more than 5 Å in a method for identifying a ligand capable of binding to a β-propeller domain of SEQ ID NO: 1; and/or a fragment or variant thereof.

31. The use according to claim 30, wherein the ligand is capable of binding to at least one amino acid residue selected from the group consisting of amino acid residues 528, 459, 468, 475 to 479, 663, and 704 of SEQ ID NO: 1.

32. The use according to claim 31, wherein the ligand is capable of binding to at least one amino acid residue, such as 1, such as 2, such as 3, such as 4, such as 5, such as 6, such as 7, such as 9, such as 10 amino acid residues selected from the group consisting of amino acid residues 528, 459, 468, 475 to 479, 663, and 704 of SEQ ID NO: 1.

33. A screening method for identification of compounds capable of inhibiting the SorCS2:progranulin binding.

34. A method of identifying a ligand capable of binding to a β-propeller domain of SEQ ID NO: 1, and/or a fragment or variant thereof, said method comprising the steps of:
a. Generating the spatial structure β-propeller domain of SEQ ID NO: 1 on a computer screen using atomic coordinates as presented in FIG. 12 or atomic coordinates selected from a three-dimensional structure that deviates from the three-dimensional structure that deviates from the three-dimensional structure presented in FIG. 12 by a root mean square deviation over protein backbone atoms of no more than 5 Å;
b. Generating potential ligands with their spatial structure on the computer screen; and
c. Selecting ligands that can bind to at least 1 amino acid residue of the set of binding interaction sites without steric interference.

35. A computer-based method for identifying a ligand capable of binding to a β-propeller domain of SEQ ID NO: 1, and/or a fragment or variant thereof, said method comprising the steps of:
a. Providing a digital embodiment of a three-dimensional structure from the atomic coordinates as presented in FIG. 12, comprising a propeller domain of SEQ ID NO: 1;
b. Generating a digital embodiment of a potential ligand in a computer; and
c. Selecting a ligand that can bind to at least one amino acid residue of the propeller domain of SEQ ID NO: 1.

36. A computer-assisted method for identifying a ligand capable of binding to a β-propeller domain of SEQ ID NO: 1 (SorCS2), and/or a fragment or variant thereof, using a programmed computer comprising a processor, a data storage system, a data input device, and a data output device, said method comprising the steps of:
a. inputting into the programmed computer through said input device data comprising:
atomic coordinates of a subset of the atoms of said SorCS2, thereby generating a criteria data set;
wherein said atomic coordinates are selected form at least part of the atomic coordinates data contained in FIG. 12 or atomic coordinates selected from a three-dimensional structure that deviates from the three-dimensional structure presented from the atomic coordinates as presented in FIG. 12, by a root mean square deviation over protein backbone atoms of not more than 5 Å;
b. comparing, using said processor, the criteria data set to a computer data base of low-molecular weight organic chemical structures and peptide fragments stored in the data storage system; and
c. selecting from said data base, using computer methods, a chemical structure having a portion that is structurally complementary to the criteria data set.

37. A method of identifying a potential ligand of a β-propeller domain of SEQ ID NO: 1, and/or a fragment or variant thereof, said method comprising the steps of:
a. introducing into a computer, information derived from atomic coordinates defining a conformation of the β-propeller domain of SEQ ID NO: 1 (SorCS2), and/or a fragment or variant thereof, based on three-dimensional structure determination, whereby a computer program utilizes or displays on the computer screen the structure of said conformation;
wherein said atomic coordinates are selected from the three-dimensional structure as presented from the atomic coordinates as presented in FIG. 12 or atomic coordinates selected from a three-dimensional structure that deviates from the three-dimensional structure presented from the atomic coordinates as presented in FIG. 12 by a root mean square deviation over protein backbone atoms of not more than 5 Å;
b. generating a three-dimensional representation of the β-propeller domain of SEQ ID NO: 1 by said computer program on a computer screen;
c. superimposing a model of a potential ligand on the representation of said β-propeller domain;
d. assessing the possibility of binding and the absence of steric interference of the potential ligand with the β-propeller domain;
e. incorporating said potential ligand compound in a binding assay of said receptor SorCS2; and
f. determining whether said potential ligand inhibit binding of a competing ligand.

38. The method according to claim 37, wherein the competing ligand is progranulin or a fragment thereof.

39. The method according to claim 38, wherein the progranulin fragment comprises the four most C-terminal amino acid residues of progranulin.

40. A method for identifying ligand of SorCS2, or a fragment thereof, said method comprising the steps of:
a. Selecting a potential ligand using atomic coordinates in conjunction with computer modelling, wherein said atomic coordinates are the atomic coordinated presented in FIG. 12 or atomic coordinates selected from a three-dimensional structure that deviates from the three-dimensional structure presented from the atomic coordinates as presented in FIG. 12 by a root mean square deviation over protein backbone atoms of not more than 5 Å, by docking potential ligands into a set of binding interaction sites in the β-propeller domain of SEQ ID NO: 1, or a fragment thereof, said binding interaction generated by computer modelling and selecting a potential ligand capable of binding to at least one amino acid residue in said set of binding interaction sites in SorCS2;
b. Providing said potential ligand and said receptor SorCs2;
c. Contacting the potential ligand with said receptor SorCS2; and
d. Detecting binding of said receptor SorCS2 by the potential ligand.

41. The method according to any one of the preceding claims, wherein the ligand of SorCS2, or a fragment thereof, is capable of binding to a β-propeller domain of SEQ ID NO: 1, and/or a fragment or variant thereof.

42. The method according to any one of the preceding claims, wherein the ligand binds to at least 1 amino acid residue of the propeller domain of SEQ ID NO: 1, such as at least 2 amino acid residues, such as at least 3 amino acid residues, such as at least 4 amino acid residues, such as at least 5 amino acid residues of the propeller domain of SEQ ID NO: 1.

43. The method according to any one of claims 34 to 42, wherein the ligand is capable of binding to at least one amino acid residue selected from the group consisting of amino acid residues 528, 459, 468, 475 to 479, 663, and 704 of SEQ ID NO: 1.

44. The method according to any one of claims 34 to 43, wherein the ligand is capable of binding to at least one amino acid residue, such as 1, such as 2, such as 3, such as 4, such as 5, such as 6, such as 7, such as 9, such as 10 amino acid residues selected from the group consisting of amino acid residues 528, 459, 468, 475 to 479, 663, and 704 of SEQ ID NO: 1.

45. The method according to any one of the preceding claims, wherein the atomic coordinates are determined to a resolution of 4.5 Å or less, such as 4 Å or less, such as 3 Å or less, such as 2 Å or less, such as 1.5 Å or less.

46. The method according to any one of the preceding claims, wherein the potential ligand is selected from the group consisting of non-hydrolyzable peptides and peptide analogues, organic compounds and inorganic compounds.

47. The method according to any one of the preceding claims, wherein a library of small organic molecules is screened.

48. An agent comprising the sequence RQLL (SEQ ID NO: 2), wherein the agent comprises no more than 20 amino acid residues.

49. The agent according to claim 48, wherein the agent is a peptide.

50. The agent according to any one of claims 48 to 50, wherein the agent comprises no more than 19 amino acid residues, such as no more than 18 amino acid residues, such as no more than 17 amino acid residues, such as no more than 16 amino acid residues, such as no more than 15 amino acid residues, such as no more than 14 amino acid residues, such as no more than 13 amino acid residues, such as no more than 12 amino acid residues, such as no more than 11 amino acid residues, such as no more than 10 amino acid residues, such as no more than 9 amino acid residues, such as no more than 8 amino acid residues, such as no more than 7 amino acid residues, such as no more than 6 amino acid residues, such as no more than 5 amino acid residues.

51. The agent according to any one of claims 48 to 51, wherein the agent comprises at least 5 amino acid residues, such as at least 6 amino acid residues, such as at least 7 amino acid residues, such as at least 8 amino acid residues, such as at least 9 amino acid residues, such as at least 10 amino acid residues, such as at least 11 amino acid residues, such as at least 12 amino acid residues, such as at least 13 amino acid residues, such as at least 14 amino acid residues, such as at least 15 amino acid residues, such as at least 16 amino acid residues, such as at least 17 amino acid residues, such as at least 18 amino acid residues, such as at least 19 amino acid residues.

52. The agent according to any one of claims 48 to 52, wherein the number of amino acid residues of the agent is in the range of 4 to 20 amino acid residues, such as 4 to 15 amino acid residues, such as 4 to 10 amino acid residues, such as 4 to 8 amino acid resides.

53. The agent according to any one of claims 48 to 53, wherein the agent comprises or consists of a peptide of the sequence RQLL (SEQ ID NO: 2).

54. An agent according to any one of claims 48 to 53 for use as a medicament.

55. An agent according to any one of claims 48 to 53 for use in the treatment of frontotemporal dementia (FTD).

56. A method for increasing the extracellular levels of progranulin, said method comprising administering an agent as defined in any one of the preceding claims.

57. Use of the agent according to any one of claims 48 to 53 for the preparation of a medicament for the treatment of frontotemporal dementia (FTD) in a subject.

58. A method for treating frontotemporal dementia (FTD), said method comprising administering an agent according to any one of claims 48 to 53 to a subject in need thereof.

59. The method according to claim 58, wherein the subject is a mammal.

60. The method according to claim 59, wherein the mammal is human.


SEQUENCE LISTING (2 sequences)

SEQ ID NO: 1 - protein, 1076 aa, Homo sapiens
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGG

SEQ ID NO: 2 - protein, 4 aa, Artificial Sequence
  synthetic peptide
  MISC_FEATURE (1)..(4): synthetic sequence
RQLL